(19) United States
(12) Patent Application Publication
(10) Pub. No.: US 2025/0312496 A1
(43) Pub. Date: Oct. 9, 2025

(54) PROSTATE SPECIFIC MEMBRANE ANTIGEN (PSMA) LIGANDS

(71) Applicants: Deutsches Krebsforschungszentrum Stiftung des öffentlichen Rechts, Heidelberg (DE); Albert-Ludwigs-Universität Freiburg, Freiburg (DE)

(72) Inventors: Matthias EDER, Heidelberg (DE); Ann-Christin EDER, Simonswald (DE); Nicolas GEIS, Freiburg (DE)

(73) Assignees: Deutsches Krebsforschungszentrum Stiftung des öffentlichen Rechts, Heidelberg (DE); Albert-Ludwigs-Universität Freiburg, Freiburg (DE)

(21) Appl. No.: 18/866,055

(22) PCT Filed: May 16, 2023
(86) PCT No.: PCT/EP2023/063102

§ 371 Date: Nov. 14, 2024

(30) Foreign Application Priority Data
May 17, 2022 (EP) 22173813.1

(51) Int. Cl. A61K 51/04

(52) U.S. Cl. CPC A61K 51/0482; A61K 51/0497

(57) ABSTRACT

The present invention generally relates to the field of radiopharmaceuticals and their use in nuclear medicine as tracers, imaging agents and for the treatment of various disease states of PSMA-expressing cancers, especially prostate cancer, and metastases thereof. In particular, the present invention relates to a PSMA binding ligand or a pharmaceutically acceptable salt or solvate thereof as well as to the use of said PSMA binding ligand, the PSMA binding ligand comprising a PSMA binding motif Q and a chelator residue A linked via at least one linker L^{AQ} comprising at least one, optionally N-alkylated, neutral amino acid Xᵢ.

### FIELD OF THE INVENTION

The present invention generally relates to the field of radiopharmaceuticals and their use in nuclear medicine as tracers, imaging agents and for the treatment of various disease states of PSMA-expressing cancers, especially prostate cancer, and metastases thereof.

### RELATED ART

Prostate cancer (PCa) is the leading cancer in the US and European population. At least 1-2 million men in the western hemisphere suffer from prostate cancer and it is estimated that the disease will strike one in six men between the ages of 55 and 85. There are more than 300,000 new cases of prostate cancer diagnosed each year in USA. The mortality from the disease is second only to lung cancer. Currently, imaging methods with high resolution of the anatomy, such as computed tomography (CT), magnetic resonance (MR) imaging and ultrasound, predominate for clinical imaging of prostate cancer. An estimated annual $2 billion is currently spent worldwide on surgical, radiation, drug therapy and minimally invasive treatments of prostate cancer. For treatment of localized prostate cancer, radical prostatectomy with lymph node dissection is an established curative strategy. However, the precise localization and delineation of tumor margins and metastases remain challenging. There is presently no effective therapy for relapsing, metastatic, androgen-independent prostate cancer.

It is well known that tumors may express unique proteins associated with their malignant phenotype or may over-express normal constituent proteins in greater number than normal cells. The expression of distinct proteins on the surface of tumor cells offers the opportunity to diagnose and characterize disease by probing the phenotypic identity and biochemical composition and activity of the tumor. Radioactive molecules that selectively bind to specific tumor cell surface proteins provide an attractive route for imaging and treating tumors under non-invasive conditions. A promising new series of low molecular weight imaging agents targets the prostate-specific membrane antigen (PSMA) (Mease R. C. et al. Clin Cancer Res. 2008, 14, 3036-3043; Foss, C. A.; et al. Clin Cancer Res 2005, 11, 4022-4028; Pomper, M. G.; et al. Mol Imaging 2002, 1, 96-101; Zhou, J.; et al. Nat Rev Drug Discov 2005, 4, 015-1026; WO 2013/022797).

A variety of experimental low molecular weight PCa imaging agents are currently being pursued clinically, including radiolabeled choline analogs [¹⁸F]fluorodihydrotestosterone ([¹⁸F]FDHT), anti-1-amino-3-[18F]fluorocyclobutyl-1-carboxylic acid (anti [¹⁸F]F-FACBC, [¹¹C]acetate and 1-(2-deoxy-2-[¹⁸F]fluoro-L-arabinofuranosyl)-5-methyluracil (-[¹⁸F]FMAU) (Scher, B.; et al. Eur J Nucl Med Mol Imaging 2007, 34, 45-53; Rinnab, L; et al. BJU Int 2007, 100, 786, 793; Reske, S. N.; et al. J Nucl Med 2006, 47, 1249-1254; Zophel, K.; Kotzerke, J. Eur J Nucl Med Mol Imaging 2004, 31, 756-759; Vees, H.; et al. BJU Int 2007, 99, 1415-1420; Larson, S. M.; et al. J Nucl Med 2004, 45, 366-373; Schuster, D. M.; et al. J Nucl Med 2007, 48, 56-63; Tehrani, O. S.; et al. J Nucl Med 2007, 48, 1436-1441). Each operates by a different mechanism and has certain advantages, e.g., low urinary excretion for [¹¹C]choline, and disadvantages, such as the short physical half-life of positron-emitting radionuclides.

PSMA is a trans-membrane, 750 amino acid type II glycoprotein that has abundant and restricted expression on the surface of PCa, particularly in androgen-independent, advanced and metastatic disease (Schulke, N.; et al. Proc Natl Acad Sci USA 2003, 100, 12590-12595). The latter is important since almost all PCa become androgen independent over the time. PSMA possesses the criteria of a promising target for therapy (Schulke, N.; et al. Proc. Natl. Acad. Sci. USA 2003, 100, 12590-12595). The PSMA gene is located on the short arm of chromosome 11 and functions both as a folate hydrolase and neuropeptidase. It has neuropeptidase function that is equivalent to glutamate carboxypeptidase II (GCPII), which is referred to as the “brain PSMA”, and may modulate glutamatergic transmission by cleaving/V-acetylaspartylglutamate (NAAG) to N-acetylaspartate (NAA) and glutamate (Nan, F.; et al. J Med Chem 2000, 43, 772-774). There are up to 10⁶ PSMA molecules per cancer cell, further suggesting it as an ideal target for imaging and therapy with radionuclide-based techniques (Tasch, J.; et al. Crit Rev Immunol 2001, 21, 249-261).

The radio-immunoconjugate of the anti-PSMA monoclonal antibody (mAb) 7E11, known as the PROSTASCINT® scan, is currently being used to diagnose prostate cancer metastasis and recurrence. However, this agent tends to produce images that are challenging to interpret (Lange, P. H. PROSTASCINT scan for staging prostate cancer. Urology 2001, 57, 402-406; Haseman, M. K.; et al. Cancer Biother Radiopharm 2000, 15, 131-140; Rosenthal, S. A.; et al. Tech Urol 2001, 7, 27-37). More recently, monoclonal antibodies have been developed that bind to the extracellular domain of PSMA and have been radiolabeled and shown to accumulate in PSMA-positive prostate tumor models in animals. However, diagnosis and tumor detection using monoclonal antibodies has been limited by the low permeability of the monoclonal antibody in solid tumors.

The selective targeting of cancer cells with radiopharmaceuticals, either for imaging or therapeutic purposes is challenging. A variety of radionuclides are known to be useful for radio-imaging or cancer radiotherapy, including ¹¹¹In, ⁹⁰Y, ⁶⁸Ga, ¹⁷⁷Lu, ^{99m}Tc, ¹²³I and ¹³¹I. Recently it has been shown that some compounds containing a glutamate-urea-glutamate (GUG) or a glutamate-urea-lysine (GUL) recognition element linked to a radionuclide-ligand conjugate exhibit high affinity for PSMA.

In WO 2015/055318 new imaging agents with improved tumor targeting properties and pharmacokinetics were described. These compounds comprise a motif specifically binding to cell membranes of cancerous cells, wherein said motif comprises a prostate-specific membrane antigen (PSMA), that is the above mentioned glutamate-urea-lysine motif. The preferred molecules described in WO 2015/055318 further comprise a linker which binds via an amide bond to a carboxylic acid group of DOTA as chelator. Some of these compounds have been shown to be promising agents for the specific targeting of prostate tumors. The compounds were labeled with ¹⁷⁷Lu (for therapy purposes) or ⁶⁸Ga (for diagnostic purposes) and allow for visualization and targeting of prostate cancer for radiotherapy purposes.

However, in therapeutic applications of radioactively labeled PSMA inhibitors, organs with physiological PSMA expression turned out to be dose limiting and thus minimize the therapeutic success. In particular, the high renal and salivary gland uptake of the radioactively labeled PSMA inhibitor substances is noticeable, which, in the case of a therapeutic application, gives rise to considerable side effects. Attempts to improve the kidney uptake of PSMA inhibitors has led to the development of PSMA-617 [Benesova, M., et al. (2016) J Med Chem 59, 1761-75], a compound which is already used clinically with 177Lu or 225Ac for endoradiotherapy of prostate cancer. However, a reduction in salivary and lacrimal gland uptake has not yet been achieved and is still described as critical and dose-limiting in early clinical work. In a first-in-man study with 225Ac-PSMA-617, two patients with extremely advanced and end-stage disease showed complete remission. In both patients the PSA value fell below the detectability limit. Accompanying diagnostic recordings with 68Ga-PSMA-11 confirmed a complete response.

As already mentioned above, the strong accumulation of PSMA ligands in non-target tissues, in particular the salivary and lacrimal glands, which has been described in numerous scientific publications leads to considerable side effects. The salivary and lacrimal glands may be severely and partially irreversibly damaged, in particular during alpha therapy with 225Ac. The resulting xerostomia for example represents a dose-limiting side effect. To resolve this issue, improvement of tissue specificity of PSMA ligands was proposed e.g. in WO 2020/165420 A1.

Nonetheless, there is still the need for improved PSMA ligands which provide advantageous options for the detection, treatment and management of PSMA-expressing cancers, in particular prostate cancer, and which preferably show less side effects and/or improved specificity in labelling and/or radiotherapy.

### SUMMARY OF THE INVENTION

The solution of said object is achieved by providing the embodiments characterized in the claims.

The inventors found new PSMA binding ligands which are useful and advantageous radiopharmaceuticals and which can be used in nuclear medicine as tracers, imaging agents and for the treatment of various disease states of PSMA-expressing cancers, in particular prostate cancer.

These PSMA binding ligands are described in more detail below:

In particular, present invention relates to PSMA binding ligand or a pharmaceutically acceptable salt or solvate thereof comprising a PSMA binding motif Q and a chelator residue A linked via at least one linker L^{AQ} comprising at least one, optionally N-alkylated, neutral amino acid X₁, wherein X₁ is preferably selected from the group consisting of, optionally N-alkylated, Gly, Ala, βAla, Phe, amino hexanoic acid (Ahx=—NH—(CH₂)₅—C(═O)—) and amino acids comprising at least one —(CH₂—CH₂—O)— group between the N-terminus and the C-terminus, or wherein X₁ is Tyr, more preferably, wherein X₁ is selected from the group consisting of Gly, Ala, βAla, Phe and amino acid residues having the structure —NH—CH₂—CH₂—O—(CH₂—CH₂—O)ₓ₁—(CH₂)_{y1}—C(═O)— with x1 being an integer in the range of from 0 to 15, and y1 being 1 or 2, or wherein X₁ is Tyr.

In particular, the present invention relates to PSMA binding ligand having the structure (I)

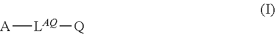

in which the PSMA binding motif Q and the chelator residue A are linked via at least one linker L^{AQ} comprising the least one neutral amino acid X₁.

Further, the present invention relates to a complex comprising
(a) a radionuclide, and
(b) the PSMA binding ligand, as described above or below, or a pharmaceutically acceptable salt or solvate thereof.

Further, the present invention relates to a pharmaceutical composition comprising a PSMA binding ligand, as described above or below, or a pharmaceutically acceptable salt or solvate thereof, as described above or below, or a complex, as described above or below.

Further, the present invention relates to a PSMA binding ligand, as described above or below, or a pharmaceutically acceptable salt or solvate thereof, or a complex, as described above or below, or a pharmaceutical composition as described above or below, for use in treating or preventing PSMA-expressing cancers, in particular prostate cancer, and/or metastases thereof

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s).

### PSMA Binding Motif

The PSMA binding motif Q has preferably the structure

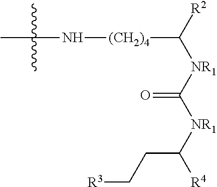

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂. More preferably, R², R³ and R⁴ are CO₂H. In particular, R¹ is H and R², R³ and R⁴ are CO₂H. The wavy line indicates the connection site to the linker L^{AQ}.

### Chelator Residue A

A is a chelator residue derived from a chelator selected from the group consisting of 1,4,7,10-tetraazacyclododecane-N,N′,N″,N′″-tetraacetic acid (=DOTA), N,N″-bis [2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl (2-carboxyethyl)phosphinic acid]-4,7-bis [methyl (2-hydroxymethyl)phosphinic acid] (NOPO), 3,6,9,15-tetraazabicyclo [9.3.1]pentadeca-1 (15),11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA)

The term “a chelator residue” and typically also the term “chelator residue derived from a chelator selected from the group” is denoted to mean that the above mentioned chelators, thus typically the chelators defined in the “group”, have been linked, via a suitable functional group, to the rest of the PSMA binding ligand, preferably to the linker L^{AQ}.

More preferably chelators defined in the “group”, have been linked, via a former carboxylic acid group of the chelator, to an N-terminal end of L^{AQ}, thereby forming an amide bond between the chelator and L^{AQ}.

Preferably, A is a chelator residue having a structure selected from the group consisting of

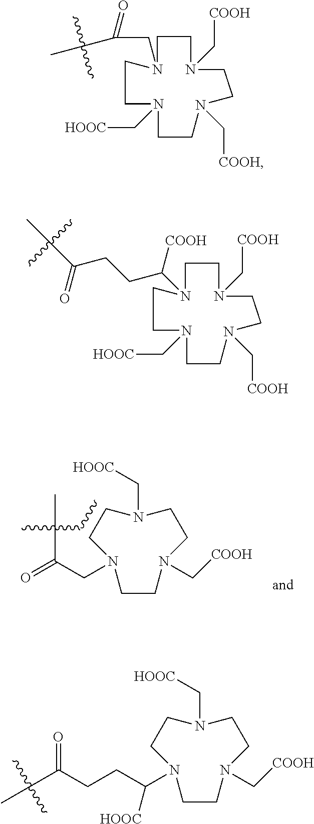

Most preferably, A has the structure

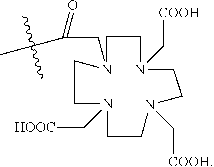 [SMILES: *C(=O)CN1CCN(CC(=O)O)CCN(CC(=O)O)CCN(CC(=O)O)CC1]

### Linker L^{AQ}

Preferably, the linker L^{AQ} comprises besides the at least one, optionally N-alkylated, neutral amino acid X₁, preferably besides the group (X₁)ₙ₁, wherein X₁ is the optionally N-alkylated, neutral amino acid X₁, and wherein n1 is in the range of from 2 to 25, further at least one amino acid building block AS^{a} and/or at least one amino acid building block AS^{B}, preferably at least one amino acid building block AS^{a} and at least one amino acid building block AS^{B}.

### Amino Acid Building Block AS^{a}

The amino acid building block AS^{a} has in particular the structure

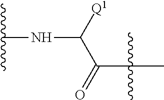

wherein Q¹ is selected from the group consisting of alkylaryl, arylalkyl, aryl, alkylheteroaryl, heteroarylalkyl and heteroaryl.

The term “aryl”, as used in this context of the invention, means optionally substituted, 5- and 6-membered aromatic rings, and substituted or unsubstituted polycyclic aromatic groups (aryl groups), for example tricyclic or bicyclic aryl groups. Optionally substituted phenyl groups or naphthyl groups may be mentioned as examples. Polycyclic aromatic groups can also contain non-aromatic rings.

The term “alkylaryl” as used in this context of the invention refers to aryl groups in which at least one proton has been replaced with an alkyl group (Alkyl-aryl-).

The term “arylalkyl” as used in this context of the invention refers to aryl groups linked via an alkyl group (Aryl-alkyl-).

The term “heteroaryl”, as used in this context of the invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzothiazolyl, benzothiophenyl, benzoxazolyl, benzodioxazolyl, benzoimidazolyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in this context of the invention refers to heteroaryl groups in which at least one proton has been replaced with an alkyl group (Alkyl-Heteroaryl-).

The term “heteroarylalkyl” as used in this context of the invention refers to heteroaryl groups linked via an alkyl group (Heteroaryl-alkyl-).

The term “cycloalkyl” means, in the context of the invention, optionally substituted, cyclic alkyl residues, wherein they can be monocyclic or polycyclic groups. Optionally substituted cyclohexyl may be mentioned as a preferred example of a cycloalkyl residue.

The term “heterocycloalkyl”, as used in this context of the invention refers to optionally substituted, cyclic alkyl residues, which have at least one heteroatom, such as O, N or S in the ring, wherein they can be monocyclic or polycyclic groups.

The terms “substituted cycloalkyl residue” or “cycloheteroalkyl”, as used in this context of the invention refers, mean cycloalkyl residues or cycloheteroalkyl residues, in which at least one H has been replaced with a suitable substituent.

Preferably, Q1 comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably Q1 is selected from the group consisting of:

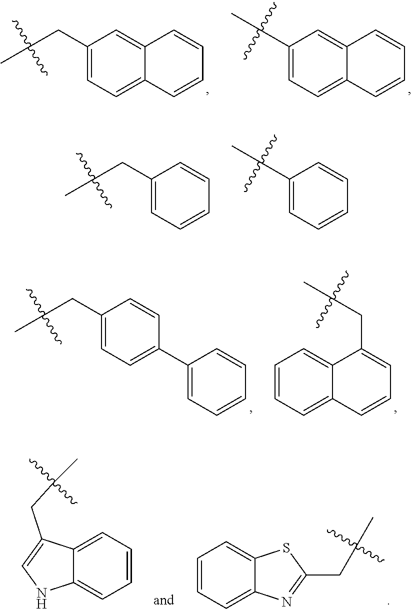

wherein Q¹ is most preferably

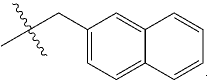 [SMILES: *Cc1ccc2ccccc2c1]

### Amino Acid Building Block AS^{b}

The amino acid building block AS^{b} preferably has the structure (b)

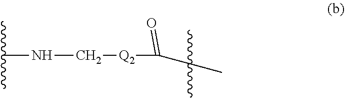

wherein Q² is selected from the group consisting of aryl, alkylaryl, arylalkyl, cycloalkyl, heterocycloalkyl, heteroaryl, heteroarylalkyl and alkylheteroaryl.

The term “aryl”, as used in this context of the invention refers to optionally substituted, 5- and 6-membered aromatic rings, and substituted or unsubstituted polycyclic aromatic groups (aryl groups), for example tricyclic or bicyclic aryl groups (—Ar—). Optionally substituted phenyl groups or naphthyl groups may be mentioned as examples. Polycyclic aromatic groups can also contain non-aromatic rings, the Aryl group in this context of the invention

The term “alkylaryl” as used in this context of the invention refers to aryl groups in which at least one proton has been replaced with an alkyl group (-alkyl-aryl-) and which are linked via to alkyl group to the —CH₂— group and via the aryl group to the carbonyl group.

The term “arylalkyl” as used in this context of the invention refers to aryl groups linked via an alkyl group to the carbonyl group and via the aryl group to the —CH₂— group (-aryl-alkyl-).

The term “heteroaryl” (-Heteraryl-, as used in this context of the invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in this context of the invention refers to aryl groups in which at least one proton has been replaced with an alkyl group (-alkyl-heteroaryl-) and which are linked via to alkyl group to the —CH₂— group and via the heteroaryl group to the carbonyl group.

The term “heteroarylalkyl” as used in this context of the invention refers to heteroaryl groups linked via an alkyl group to the carbonyl group and via the heteroaryl group to the —CH₂— group (-aryl-alkyl-).

The term “cycloalkyl” (-cycloalkyl-) means, in the context of the invention, optionally substituted, cyclic alkyl residues, wherein they can be monocyclic or polycyclic groups. Optionally substituted cyclohexyl may be mentioned as a preferred example of a cycloalkyl residue.

The term “heterocycloalkyl”, as used in this context of the invention refers to optionally substituted, cyclic alkyl residues, which have at least one heteroatom, such as O, N or S in the ring, wherein they can be monocyclic or polycyclic groups.

The terms “substituted cycloalkyl residue” or “cycloheteroalkyl”, as used in this context of the invention refers, mean cycloalkyl residues or cycloheteroalkyl residues, in which at least one H has been replaced with a suitable substituent.

Preferably, Q² is an aryl group or cycloalkyl group, more preferably

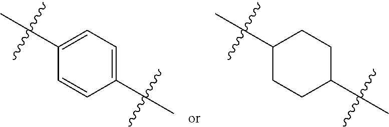 [SMILES: *c1ccc(*)cc1 ; *C1CCC(*)CC1]

most preferably

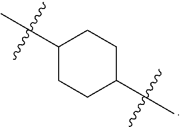 [SMILES: *C1CCC(*)CC1]

It is to be understood that any stereoisomers of Q2 are possibly and included. In case Q2 is

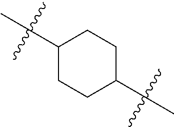 [SMILES: *C1CCC(*)CC1]

it is to be understood that this includes the cis as well as the trans isomer, with the trans isomer being particularly preferred.

The PSMA binding ligand, described above or below, has preferably a structure selected from the group consisting of (Ia), (Ib) and (Ic)

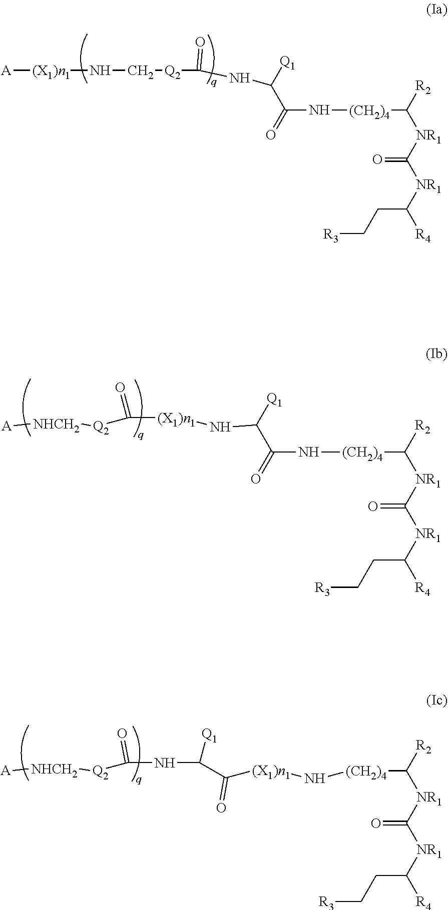

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂,
Q¹ is selected from the group consisting of alkylaryl, arylalkyl, aryl, alkylheteroaryl, heteroarylalkyl and heteroaryl,
Q² is selected from the group consisting of aryl, alkylaryl, arylalkyl, cycloalkyl, heterocycloalkyl, heteroaryl, heteroarylalkyl and alkylheteroaryl,
and wherein q is an integer of from 0-3, preferably 1, and wherein X₁ and n1 are as described above and below.

The PSMA binding ligand, described above or below, has more preferably the structure (Ia)

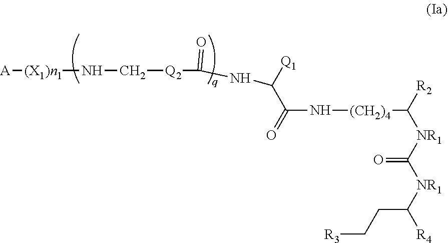

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂,
Q¹ is selected from the group consisting of alkylaryl, arylalkyl, aryl, alkylheteroaryl, heteroarylalkyl and heteroaryl,
Q² is selected from the group consisting of aryl, alkylaryl, arylalkyl, cycloalkyl, heterocycloalkyl, heteroaryl, heteroarylalkyl and alkylheteroaryl,
and wherein q is an integer of from 0-3, preferably 1, and wherein X₁ and n1 are as described above and below.

More preferably the PSMA binding ligand, described above or below, has the structure (Ia), wherein Q¹ comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

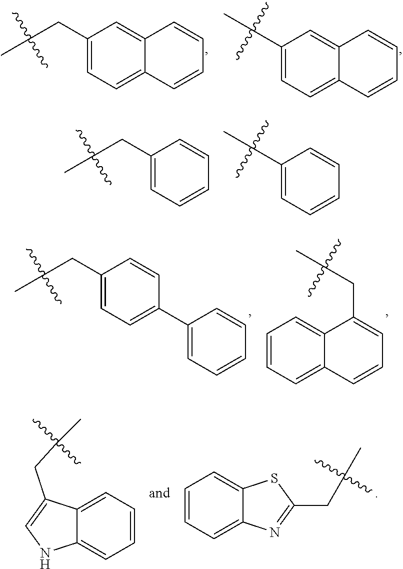

preferably wherein Q¹ is

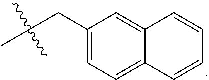 [SMILES: *Cc1ccc2ccccc2c1]

and wherein R³, R² and R⁴ are preferably —CO₂H and R¹ is H., and wherein Q² is preferably

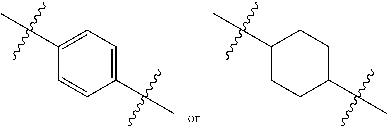 [SMILES: *c1ccc(*)cc1 ; *C1CCC(*)CC1]

more preferably

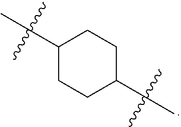 [SMILES: *C1CCC(*)CC1]

In particular, the present invention relates to a PSMA binding ligand, as described above and below, the ligand having the structure

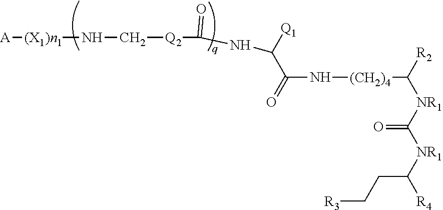

wherein A is a chelator residue having the structure

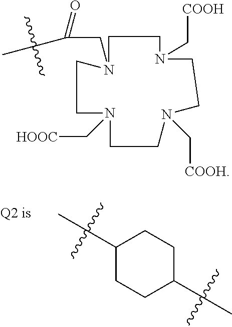 [SMILES: *C(=O)CN1CCN(CC(=O)O)CCN(CC(=O)O)CCN(CC(=O)O)CC1 ; *C1CCC(*)CC1]

more preferably

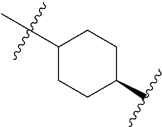

q is 1
Q¹ is

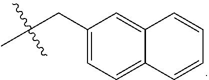 [SMILES: *Cc1ccc2ccccc2c1]

and wherein R³, R² and R⁴ are —CO₂H and R¹ is H and wherein n1 is preferably in the range of from 5 to 25, preferably 3 to 10,
X₁ and n1 are as described above and below.

### The Neutral Amino Acid X₁

As described above, the least one linker L^{AQ} comprises at least one neutral amino acid. The term “neutral amino acid” as used within the meaning of the present invention includes each and every amino acid having no net charge at a pH of 7. It is to be understood that the term includes all naturally-occurring and non-naturally-occurring amino acids, including all stereoisomers, such as enantiomers and diastereomers of these amino acids, such as glycine, alanine, valine, isoleucine, phenylalanine, beta-alanine as well as unnatural amino acids comprising a neutral linker between N and C terminus, such at least one —(CH2-CH2-O)— group between the N-terminus and the C-terminus, as well as N-alkylated such as N-methylated amino acids, thus amino acids comprising instead of a proton-H an alkyl or Methyl group attached to the amino group of the amino acid.

N-alkylated amino acids, include, but are not limited to, N-alkyl-glycine, N-alkyl-alanine, N-alkyl-valine, N-alkyl-isoleucine, N-alkyl-leucine, N-alkyl-methionine, N-alkyl-phenylalanine, N-alkyl-tyrosine or N-alkyl-tryptophane. N-methyl amino acids include, but are not limited to N-methyl amino acid is selected from the group consisting of N-methyl glycine, N-methyl alanine, N-methyl valine, N-methyl isoleucine, N-methyl leucine, N-methyl phenylalanine,

If the amino acid is chiral, the amino acid preferably has L-conformation.

Preferably, the neutral amino acid X₁ is selected from the group consisting of, optionally N-alkylated, Gly, Ala, βAla, Phe, amino hexanoic acid (Ahx=—NH—(CH₂)₅—C(═O)—) and amino acids comprising at least one —(CH2-CH2-O)— group between the N-terminus and the C-terminus, more preferably, wherein X₁ is selected from the group consisting of Gly, Ala, βAla, Phe and amino acid residues having the structure —NH—CH₂—CH₂—O—(CH₂—CH₂—O)ₓ₁—(CH₂)_{y1}—C(═O)— with x1 being an integer in the range of from 0 to 15, and y1 being 1 or 2.

According to a further preferred embodiment, the amino acid is, optionally N-alkylated, tyrosine (Tyr).

Optionally, the linker L^{AQ}, comprises besides the optionally present at least one amino acid building block AS^{a} and the optionally present at least one amino acid building block AS^{B}, and besides X₁, further amino acids X, wherein preferably all additional amino acids X are neutral amino acids.

According to one preferred embodiment, the linker L^{AQ} comprises an amino acid sequence AA of 2 to 25 amino acids, with amino acid X₁ being part of the sequence, and wherein preferably all amino acids present in the sequence are neutral amino acids. It is to be understood, that the sequence AA comprises the at least one amino acid X₁. Preferably, all amino acids X present in the sequence are the same and are X₁.

### The Linking Unit —(X)n₁

Preferably, the linker L^{AQ} comprises an amino acid sequence AA comprising the building block (X₁)ₙ₁, wherein n1 is in the range of from 1 to 25, such as 2 to 25. Preferably, the amino acid sequence AA consist of the block (X₁)ₙ₁, wherein all amino acids X₁ are the same.

Preferably, X₁ is selected from the group consisting of, optionally N-alkylated, Gly, Ala, βAla, Phe, amino hexanoic acid (Ahx=—NH—(CH₂)₅—C(═O)—) and amino acid residues having the structure —NH—CH₂—CH₂—O—(CH₂—CH₂—O)ₓ₁—(CH₂)_{y1}—C(═O)— with x1 being an integer in the range of from 0 to 15, and y1 being 1 or 2. If the linker comprises more than 1 group X₁, the amino acids may be the same or maybe different, preferably the same. Preferably, X₁ is not N-alkylated.

According to an alternative the linker L^{AQ} comprises besides the optionally present at least one amino acid building block AS^{a} and the optionally present at least one amino acid building block AS^{B}, and besides one amino acid X₁, no further amino acid. In this case, X₁ is preferably —NH—CH₂—CH₂—O—(CH₂—CH₂—O)ₓ₁—(CH₂)_{y1}—C(═O)— with x1 being an integer in the range of from 0 to 15, and y1 being 1 or 2 or amino hexanoic acid (Ahx=—NH—(CH₂)₅—C(═O)—).

Preferably the linker L^{AQ} comprises an amino acid sequence AA comprising the building block —(X₁)ₙ₁—, with X₁ being a neutral amino acid, more preferably with X₁ being selected from the group consisting of Gly, Ala, βAla, Phe, amino hexanoic acid (Ahx=—NH—(CH₂)₅—C(═O)—) and amino acids comprising at least one —(CH₂—CH₂—O)— group between the N-terminus and the C-terminus preferably, more preferably wherein X₁ is selected from the group consisting of Gly, Ala, βAla, Phe and amino acid residues having the structure —NH—CH₂—CH₂—O—(CH₂—CH₂—O)ₓ₁—(CH₂)_{y1}—C(═O)— with x1 being an integer in the range of from 0 to 15, and y1 being 1 or 2. Integer n1 is preferably in the range of from 1 to 25, preferably in the range of from 1 to 20.

According to a first preferred embodiment, the linker L^{AQ} comprises at least one group X₁ with X₁ being selected from the group consisting of Gly, Ala, βAla and Phe.

More preferably, the linker L^{AQ} comprises the group (X₁)ₙ₁, wherein X₁ is selected from the group consisting of Gly, Ala, βAla and Phe and n1 is in the range of from 2 to 20, preferably 3 to 15, such as 3, 4, 5, 6, 7, 8, 9, 10, 11, 12, 13, 14, 15, more preferably, 3, 5, 10 or 15.

Thus, according to a preferred embodiment, the linker L^{AQ} comprises the group (X₁)ₙ₁, wherein X₁ is selected from the group consisting of Gly, Ala, βAla and Phe and n1 is 3, more preferably wherein X₁ is Gly or Ala and n1 is 3. Thus, in a preferred embodiment, X₁ is Gly and n1 is 3. In a further preferred embodiment, X₁ is Ala and n1 is 3.

Thus, according to a preferred embodiment, the linker L^{AQ} comprises the group (X₁)ₙ₁, wherein X₁ is selected from the group consisting of Gly, Ala, βAla and Phe and n1 is 5, more preferably, X₁ is Ala, and n1 is 5.

Thus, according to a preferred embodiment, the linker L^{AQ} comprises the group (X₁)ₙ₁, wherein X₁ is selected from the group consisting of Gly, Ala, βAla and Phe and n1 is 10.

Thus, according to a preferred embodiment, the linker L^{AQ} comprises the group (X₁)ₙ₁, wherein X₁ is selected from the group consisting of Gly, Ala, βAla and Phe and n1 is 15.

According to a further preferred embodiment, the linker L^{AQ} comprises at least one group X₁ with X₁ being Gly, more preferably, the PSMA ligand comprises the group (X₁)ₙ₁, wherein X₁ is Gly and n1 is in the range of from 2 to 20, preferably 3 to 15, such as 3, 4, 5, 6, 7, 8, 9, 10, 11, 12, 13, 14, 15, more preferably, 3, 5, 10 or 15, more preferably 3.

According to a further preferred embodiment, the linker L^{AQ} comprises at least one group X₁ with X₁ being Ala, more preferably, the PSMA ligand comprises the group (X₁)ₙ₁, wherein X₁ is Ala and n1 is in the range of from 2 to 20, preferably 3 to 15, such as 3, 4, 5, 6, 7, 8, 9, 10, 11, 12, 13, 14, 15, more preferably, 3, 5, 10 or 15, more preferably 3 or 5, most preferably 5.

According to a further preferred embodiment, the linker L^{AQ} comprises at least one group X₁ with X₁ being βAla, more preferably, the PSMA ligand comprises the group (X₁)ₙ₁, wherein X₁ is βAla and n1 is in the range of from 2 to 20, preferably 3 to 15, such as 3, 4, 5, 6, 7, 8, 9, 10, 11, 12, 13, 14, 15, more preferably, 3, 5, 10 or 15.

According to a further preferred embodiment, the linker L^{AQ} comprises at least one group X₁ with X₁ being Phe, more preferably, the PSMA ligand comprises the group (X₁)ₙ₁, wherein X₁ is selected Phe and n1 is in the range of from 2 to 20, preferably 3 to 15, such as 3, 4, 5, 6, 7, 8, 9, 10, 11, 12, 13, 14, 15, more preferably, 3, 5, 10 or 15, more preferably 3 or 5.

According to a further preferred embodiment, the linker L^{AQ} comprises at least one group X₁ with X₁ being Tyr, more preferably, the PSMA ligand comprises the group (X₁)ₙ₁, wherein X₁ is Tyr and n1 is in the range of from 2 to 20, preferably 3 to 15, such as 3, 4, 5, 6, 7, 8, 9, 10, 11, 12, 13, 14, 15, more preferably, 3, 5, 10 or 15, more preferably 3 or 5, mot preferably 3.

According to a second preferred embodiment, the linker L^{AQ} comprises the group (X₁)ₙ₁, with X₁ having the structure —NH—CH₂—CH₂—O—(CH₂—CH₂—O)ₓ₁—(CH₂)_{y1}—C(═O)— and with integer x1 being in the range of from 1 to 15, and y1 being 1 or 2 and with n1 being 1. Preferably, with integer x1 being 1, 2, 3, 4, 5, 6, 7, 8, 9, 10, 11, 12, 13, 14 or 15, more preferably 1, 3, 5, 7.

According to a preferred embodiment, the linker L^{AQ} comprises the group (X₁)ₙ₁, with X₁ having the structure —NH—CH₂—CH₂—O—(CH₂—CH₂—O)ₓ₁—(CH₂)_{y1}—C(═O)— and with integer x1 being in the range of from 1 to 15, preferably 1 to 7, and with y1 being 1 or 2, preferably with y1 being 1. In a preferred embodiment, L^{AQ} comprises the group (X₁)ₙ₁, with X₁ having the structure —NH—CH₂—CH₂—O—(CH₂—CH₂—O)ₓ₁—(CH₂)_{y1}—C(═O)— with integer x1 being 1 and with y1 being 1.

According to a further preferred embodiment, the linker L^{AQ} comprises the group (X₁)ₙ₁, with X₁ having the structure —NH—CH₂—CH₂—O—(CH₂—CH₂—O)ₓ₁—(CH₂)_{y1}—C(═O)— and with integer x1 being 3 and y1 being 2.

According to a further preferred embodiment, the linker L^{AQ} comprises the group (X₁)ₙ₁, with X₁ having the structure —NH—CH₂—CH₂—O—(CH₂—CH₂—O)ₓ₁—(CH₂)_{y1}—C(═O)— and with integer x1 being 5 and y1 being 2.

According to a further preferred embodiment, the linker L^{AQ} comprises the group (X₁)ₙ₁, with X₁ having the structure —NH—CH₂—CH₂—O—(CH₂—CH₂—O)ₓ₁—(CH₂)_{y1}—C(═O)— and with integer x1 being 7 and y1 being 2.

According to a third preferred embodiment, the linker L^{AQ} comprises the group (X₁)ₙ₁, with X₁ being amino hexanoic acid and with n1 being preferably 1 to 20, preferably 1 to 15, more preferably 1 to 4, such as 1, 2, 3 or 4.

According to a preferred embodiment, the linker L^{AQ} comprises the group (X₁)ₙ₁, with X₁ being amino hexanoic acid and with n1 being 1.

According to a further preferred embodiment, the linker L^{AQ} comprises the group (X₁)ₙ₁, with X₁ being amino hexanoic acid and with n1 being 2.

According to a further preferred embodiment, the linker L^{AQ} comprises the group (X₁)ₙ₁, with X₁ being amino hexanoic acid and with n1 being 3.

According to a further preferred embodiment, the linker L^{AQ} comprises the group (X₁)ₙ₁, with X₁ being amino hexanoic acid and with n1 being 4.

By way of example, the particularly preferred PSMA binding ligands of the invention are shown in Table 1:

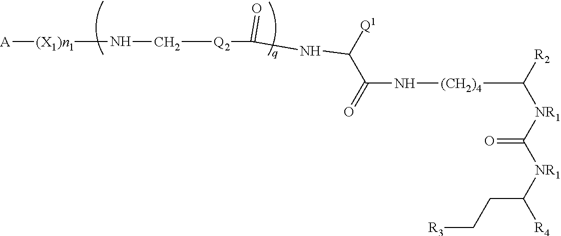

| | | | | | R3, R2 |
|---|---|---|---|---|---|
| A | (X1)n1 | Q2 | Q1 and q | R1 | and R4 |
| DOTA | Selected from the group consisting of (Gly)₃, (Gly)₅, Gly)₁₀ and (Gly)₁₅ | 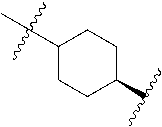 | 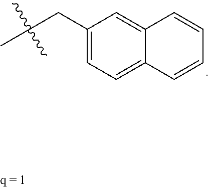 [SMILES: *Cc1ccc2ccccc2c1] | H | CO2H |
| DOTA | Selected from the group consisting of (Ala)₃, (Ala)₅, (Ala)₁₀ and (Ala)₁₅ | 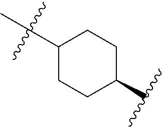 | 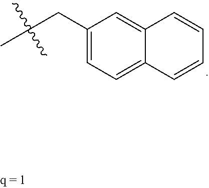 [SMILES: *Cc1ccc2ccccc2c1] | H | CO2H |
| DOTA | Selected from the group consisting of (Phe)₃, and (Phe)₁₅ | 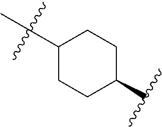 | 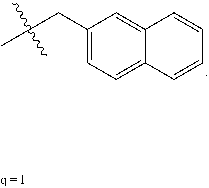 [SMILES: *Cc1ccc2ccccc2c1] | H | CO2H |
| DOTA | Selected from the group consisting of (βAla)₃, (βAla)₅, (βAla)₁₀ and (βAla)₁₅ | 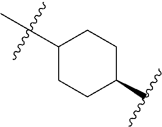 | 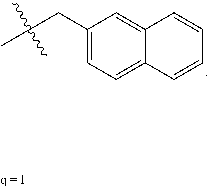 [SMILES: *Cc1ccc2ccccc2c1] | H | CO2H |
| DOTA | Selected from the group consisting of (Gly)₃ (βAla)₃, (Ala)₃ and (Phe)₃ | 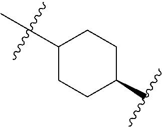 | 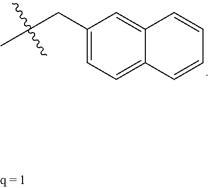 [SMILES: *Cc1ccc2ccccc2c1] | H | CO2H |
| DOTA | Selected from the group consisting of (Gly)₅ (βAla)₅, (Ala)₅ and (Phe)₅ | 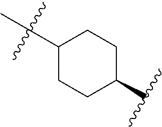 | 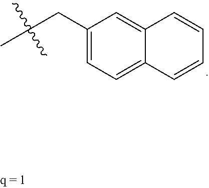 [SMILES: *Cc1ccc2ccccc2c1] | H | CO2H |
| DOTA | Selected from the group consisting of (Gly)₁₀ (βAla)₁₀, (Ala)₁₀ and (Phe)₁₀ | 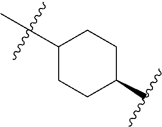 | 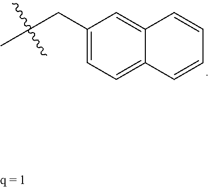 [SMILES: *Cc1ccc2ccccc2c1] | H | CO2H |
| DOTA | Selected from the group consisting of (Gly)₁₅ (βAla)₁₅, (Ala)₁₅ and (Phe)₁₅ | 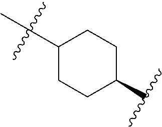 | 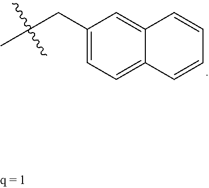 [SMILES: *Cc1ccc2ccccc2c1] | H | CO2H |
| DOTA | Selected from the group consisting of (Gly)₁₅ (βAla)₁₅, (Ala)₁₅ and (Phe)₁₅ | 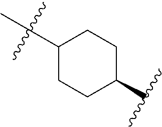 | 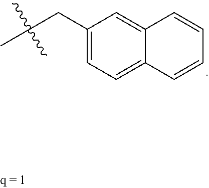 [SMILES: *Cc1ccc2ccccc2c1] | H | CO2H |
| DOTA | —NH—CH₂—CH₂—O— (CH₂—CH₂—O)ₓ₁— (CH2)_{y1}—C(═O)— with x1 being 1 and y1 being 1 | 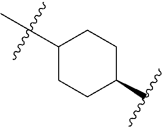 | 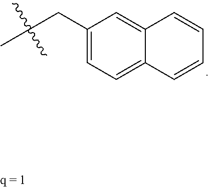 [SMILES: *Cc1ccc2ccccc2c1] | H | CO2H |
| DOTA | —NH—CH₂—CH₂—O— (CH₂—CH₂—O)ₓ₁— (CH2)_{y1}—C(═O)— with x1 being 3, 5 or 7 and y1 being 2 | 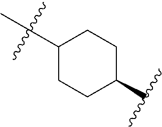 | 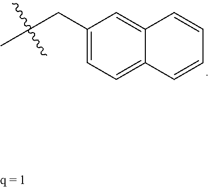 [SMILES: *Cc1ccc2ccccc2c1] | H | CO2H |
| DOTA | Selected from the group consisting of (Ahx)₁ (Ahx)₂, (Ahx)₃ and (Ahx)₄ | 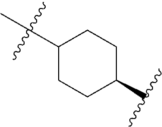 | 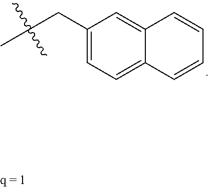 [SMILES: *Cc1ccc2ccccc2c1] | H | CO2H |
| DOTA | (Tyr)3 or (Tyr)5 | 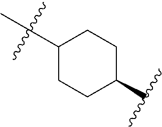 | 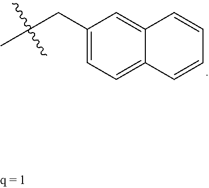 [SMILES: *Cc1ccc2ccccc2c1] | H | CO2H |

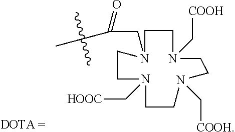 [SMILES: *C(=O)CN1CCN(CC(=O)O)CCN(CC(=O)O)CCN(CC(=O)O)CC1]

In particular, the PSMA binding ligand has the structure as shown in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, FIG. 18, FIG. 19, FIG. 20, FIG. 21 or FIG. 22, wherein preferably all chiral amino acids (X₁) are present in L-configuration.

In a preferred embodiment, the PSMA binding ligand has the structure as shown in FIG. 1, FIG. 2, FIG. 5, FIG. 6, FIG. 13, FIG. 14, FIG. 15, FIG. 16, FIG. 17, FIG. 18, FIG. 19, FIG. 20, FIG. 21 or FIG. 22.

In a particular preferred embodiment, the PSMA binding ligand has the structure as shown in FIG. 1, FIG. 5, FIG. 6, FIG. 13, FIG. 14, FIG. 15, FIG. 16, FIG. 17, FIG. 18, FIG. 19, FIG. 20, FIG. 21 or FIG. 22, more preferably the PSMA binding ligand has the structure as shown in FIG. 1, FIG. 5, FIG. 6, FIG. 15, FIG. 16, FIG. 17, FIG. 18, FIG. 19, FIG. 20, FIG. 21 or FIG. 22.

As described above, the present invention also relates to a complex comprising
(a) a radionuclide, and
(b) a PSMA binding ligand, as described above or below, or a pharmaceutically acceptable salt or solvate thereof.

Typical pharmaceutically acceptable salts include those salts prepared by reaction of the PSMA binding ligands of the present invention with a pharmaceutically acceptable mineral or organic acid or an organic or inorganic base. Such salts are known as acid addition and base addition salts. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, napththalene-2-sulfonate, mandelate and the like. Preferred pharmaceutically acceptable acid addition salts are those formed with mineral acids such as hydrochloric acid and hydrobromic acid, and those formed with organic acids such as maleic acid and methanesulfonic acid. Salts of amine groups may also comprise quaternary ammonium salts in which the amino nitrogen carries a suitable organic group such as an alkyl, alkenyl, alkynyl, or aralkyl moiety. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred. It should be recognized that the particular counter ion forming a part of any salt of this invention is usually not of a critical nature, so long as the salt as a whole is pharmacologically acceptable and as long as the counter ion does not contribute undesired qualities to the salt as a whole.

The term “pharmaceutically acceptable solvate” encompasses also suitable solvates of the PSMA binding ligands of the invention, wherein the PSMA binding ligand combines with a solvent such as water, methanol, ethanol, DMSO, acetonitrile or a mixture thereof to form a suitable solvate such as the corresponding hydrate, methanolate, ethanolate, DMSO solvate or acetonitrilate.

### The Radionuclide

Depending on whether the PSMA binding ligands of the invention are to be used as radio-imaging agents or radio-pharmaceuticals different radionuclides are complexed to the chelator.

The complexes of invention may contain one or more radionuclides, preferably one radionuclide. These radionuclides are preferably suitable for use as radio-imaging agents or as therapeutics for the treatment of proliferating cells, for example, PSMA expressing cancer cells, in particular PSMA-expressing prostate cancer cells. According to the present invention they are called “metal complexes” or “radiopharmaceuticals”.

Preferred imaging methods are positron emission tomography (PET) or single photon emission computed tomography (SPECT).

Preferably, the at least one radionuclide is selected from the group consisting ⁸⁹Zr, ⁴⁴Sc, ¹¹¹In, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb).

More preferably, the at least one radionuclide is selected from the group consisting ⁹⁰Y, ⁶⁸Ga, ¹⁷⁷Lu, ²²⁵Ac, and ²¹³Bi. More preferably, the radionuclide is ¹⁷⁷Lu or ²²⁵Ac.

Preferably, the radionuclide has a half-life of at least 30 min, more preferably of at least 1 h, more preferably at least 12 h, even more preferably at least 1d, most preferably at least 5 d; also preferably, the radionuclide has a half-life of at most 1 year, more preferably at most 6 months, still more preferably at most 1 month, even more preferably at most 14 d. Thus, preferably, the radionuclide has a half-life of from 30 min to 1 year, more preferably of 12 h to 6 months, even more preferably of from 1 d to 1 month, most preferably of from 5 d to 14 d.

Preferably, the radionuclide is an α- and/or β-emitter, i.e. the radionuclide preferably emits α-particles (α-emitter) and/or β-radiation (β-emitter).

Preferably, in case the radionuclide is an α-emitter, the α-particle has an energy of from 1 to 10 MeV, more preferably of from 2 to 8 MeV, most preferably of from 4 to 7 MeV.

Preferably, in case the radionuclide is a β-emitter, the β-radiation has an energy of from 0.1 to 10 MeV, more preferably of from 0.25 to 5 MeV, most preferably of from 0.4 to 2 MeV.

Preferred radionuclides emitting β-radiation are selected from the group consisting of ⁹⁰Y, ¹⁷⁷Lu, ⁵⁹Fe, ⁶⁶Cu, ⁶⁷Cu, ¹⁶¹Tb, ¹⁵³Sm, ²¹²Pb, ²¹¹Pb, ²¹³Pb, ²¹⁴Pb, ²⁰⁹Pb Very preferred radionuclides emitting β-radiation are ¹⁷⁷Lu or ⁹⁰Y, most preferably ¹⁷⁷Lu. Preferably in this case the use is diagnosis or therapy.

Preferred radionuclides emitting α-radiation are e.g. selected from the group consisting of ²¹³Bi, ²²⁵Ac, ¹⁴⁹Tb, ²³⁰U and ²²³Ra, ²¹³Bi, ²³⁰U, more preferably the radionuclide is ²²⁵Ac and/or ²¹³Bi. A very preferred radionuclide emitting α-radiation is e.g. ²²⁵Ac. Preferably, in this case the use is therapy.

According to a further embodiment, the radionuclide is a positron emitter. In this case the radionuclide is preferably selected from the group consisting ⁸⁹Zr, ⁴⁴Sc, ⁶⁶Ga, ⁶⁸Ga and ⁶⁴Cu. In this case, the use is preferably PET diagnosis.

According to a further preferred embodiment, radionuclide is a gamma emitter. In this case the radionuclide is preferably selected from the group consisting ¹¹¹In, ⁶⁷Ga, ^{99m}Tc, ¹⁵⁵Tb, ¹⁶⁵Er and ²⁰³Pb. In this case, the use preferably is SPECT diagnosis.

According to a further preferred embodiment, the radionuclide emits Auger electrons, and preferably decays by electron capture. In this case, the radionuclide is preferably selected from the group consisting of ⁶⁷Ga, ¹⁵⁵Tb, ¹⁵³Gd, ¹⁶⁵Er and ²⁰³Pb. In this case, the use is preferably therapy.

### Pharmaceutical Composition

As described above, the present invention also relates to a pharmaceutical composition comprising the PSMA binding ligand as described above or below, or a complex as described above or below. It is to be understood that the pharmaceutical compositions preferably comprise therapeutically effective amounts of the PSMA binding ligand and/or the complex, respectively. The pharmaceutical composition may further comprise at least one organic or inorganic solid or liquid and/or at least one pharmaceutically acceptable carrier.

The terms “medicament” and “pharmaceutical composition”, as used herein, relate to the PSMA binding ligands and/or complexes of the present invention and optionally one or more pharmaceutically acceptable carrier, i.e. excipient. The PSMA binding ligands of the present invention can be formulated as pharmaceutically acceptable salts; salts have been described herein above. The pharmaceutical compositions are, preferably, administered locally (e.g. intra-tumorally), topically or systemically. Suitable routes of administration conventionally used for drug administration are oral, intravenous, or parenteral administration as well as inhalation. A preferred route of administration is parenteral administration. A “parenteral administration route” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Preferably, administration is by intravenous administration or infusion. However, depending on the nature and mode of action of a PSMA binding ligand, the pharmaceutical compositions may be administered by other routes as well.

Moreover, the PSMA binding ligands can be administered in combination with other drugs either in a common pharmaceutical composition or as separated pharmaceutical compositions wherein said separated pharmaceutical compositions may be provided in form of a kit of parts. The PSMA binding ligands are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The excipient(s) must be acceptable in the sense of being compatible with the other ingredients of the formulation and, within the scope of sound medical judgment, suitable for use in contact with the tissues of a patient without excessive toxicity, irritation, allergic response, or other problem or complication, commensurate with a reasonable benefit/risk ratio. Preferably, an excipient is being not deleterious to the recipient thereof. The excipient employed may be, for example, a solid, a gel or a liquid carrier. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, non-immunogenic stabilizers and the like. When solutions for infusion or injection are used, they are preferably aqueous solutions or suspensions, it being possible to produce them prior to use, e.g. from lyophilized preparations which contain the active substance as such or together with a carrier, such as mannitol, lactose, glucose, albumin and the like. The readymade solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

A therapeutically effective dose refers to an amount of the PSMA binding ligands to be used in a pharmaceutical composition of the present invention which prevents, ameliorates or treats the symptoms accompanying a disease or condition referred to in this specification. Therapeutic efficacy and toxicity of such PSMA binding ligands can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age, the particular PSMA binding ligand to be administered, sex, time and route of administration, general health, and other drugs being administered concurrently. Progress can be monitored by periodic assessment. Preferred doses are specified herein below. Progress can be monitored by periodic assessment. The pharmaceutical compositions and formulations referred to herein are administered at least once in order to treat or prevent a disease or condition recited in this specification. However, the said pharmaceutical compositions may be administered more than one time, for example from one to ten times. Preferably, the pharmaceutical compositions may be administered at a frequency of once every one to six months, more preferably once every two to four months. Specific pharmaceutical compositions are prepared in a manner well known in the pharmaceutical art and comprise at least one active PSMA binding ligand referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adapted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

The term “patient”, as used herein, relates to a vertebrate, preferably a mammalian animal, more preferably a human, monkey, cow, horse, cat or dog. Preferably, the mammal is a primate, more preferably a monkey, most preferably a human.

The dosage of the PSMA binding ligand administered to a patient, preferably, is defined as a compound dosage, i.e. the amount of PSMA binding ligand administered to the patient. Preferred diagnostic compound dosages are total doses of 1-10 nmol/patient; thus, preferably, the diagnostic compound dosage is of from 0.02 to 0.1 nmol/kg body weight. Preferred therapeutic compound dosages are total doses of 10 to 100 nmol/patient; thus, preferably, the therapeutic compound dosage is of from 0.2 to 1 nmol/kg body weight.

As will be understood by the skilled person, the dosage of the complex as specified herein, i.e. a complex comprising, preferably consisting of, a radionuclide and a PSMA binding ligands, preferably is indicated as compound dosage as specified above, preferred dosages being the same as specified above. More preferably, the dosage of the complex is indicated as activity dosage, i.e. as the amount of radioactivity administered to the patient. Preferably, the activity dosage is adjusted such as to avoid adverse effects as specified elsewhere herein. Preferably, a patient-specific dose, preferably a patient-specific activity dosage, is determined taking into account relevant factors as specified elsewhere herein, in particular taking into account therapeutic progress and/or adverse effects observed for the respective patient. Thus, preferably, the activity dosage is adjusted such that the organ-specific dose in salivary glands is at most 30 Sv, more preferably less than 20 Sv, still more preferably less than 10 Sv, most preferably less than 5 Sv.

The effective amount may be administered once (single dosage) with an activity dosage of from about 2 MBq to about 30 MBq, preferably 4 to 30 Mbq, more preferably 6 to 30 Mbq, more preferably 8 to 30 Mbq, more preferably 10 to 30 Mbq, more preferably 15 to 30 Mbq, preferably 20 to 30 Mbq to the patient. Thus, a preferred therapeutic dose in such case is of from 2 MBq to about 30 MBq/patient, preferably 4 to 30 Mbq/patient, more preferably 6 to 30 Mbq/patient, more preferably 8 to 30 Mbq/patient, more preferably 10 to 30 Mbq/patient, more preferably 15 to 30 Mbq/patient, preferably 20 to 30 Mbq/patient. Preferably said activity dosage ranges from about 10 to 30 MBq per administration, such as for example about 10, 11, 12, 13, 14, 15, 16, 17, 18, 19, 20, 21, 22, 23, 24, 25, 26, 27, 28, 29 or 30 MBq, or any range between any two of the above values. However, as specified herein below, depending on the type of radiation emitted by the radionuclide and/or on the application, higher or lower doses may be envisaged. The phrases “effective amount” or “therapeutically-effective amount” as used herein mean that amount of a PSMA binding ligand, material, or composition comprising a PSMA binding ligand of the invention, or other active ingredient which is effective for producing some desired therapeutic effect in at least a sub-population of cells in a patient at a reasonable benefit/risk ratio applicable to any medical treatment. A therapeutically effective amount with respect to a PSMA binding ligand of the invention means that amount of therapeutic agent alone, or in combination with other therapies, that provides a therapeutic benefit in the treatment or prevention of a disease. Used in connection with a PSMA binding ligand of the invention, the term can encompass an amount that improves overall therapy, reduces or avoids symptoms or causes of disease, or enhances the therapeutic efficacy of or synergies with another therapeutic agent.

According to a preferred embodiment, the radionuclide is a β-emitter as specified herein above, more preferably is ¹⁷⁷Lu and the use is diagnosis; in such case, the activity dosage of the complex preferably is at least 100 kBq/kg body weight, more preferably at least 500 kBq/kg body weight, most preferably at least 1 MBq/kg body weight. More preferably the radionuclide is a β-emitter as specified herein above, more preferably is ¹⁷⁷Lu and the use is therapy, preferably therapy of prostate carcinoma as specified elsewhere herein; in such case, the activity dosage of the complex preferably is at least 25 MBq/kg body weight, more preferably at least 50 MBq/kg body weight, most preferably at least 80 MBq/kg body weight. Thus, a preferred therapeutic dose in such case is of from 2 to 10 Gbq/patient, more preferably of from 4 to 8 GBq/patient, most preferably is about 6 GBq/patient.

More preferably, the radionuclide is an α-emitter as specified herein above, more preferably is ²²⁵Ac and the use is therapy, preferably therapy of prostate carcinoma as specified elsewhere herein; in such case, the activity dosage of the complex is preferably in the range of from 25 kBq/kg to about 500 kBq/kg of body weight of said patient, more preferably, the activity dosage of the complex is at least 75 kBq/kg body weight, more preferably at least 100 kBq/kg body weight, still more preferably at least 150 kBq/kg body weight, most preferably at least 200 kBq/kg body weight. Thus, preferably, in such case, the activity dosage of the complex is of from 75 to 500 kBq/kg body weight, more preferably of from 100 to 400 kBq/kg body weight, still more preferably of from 150 to 350 kBq/kg body weight, most preferably of from 200 to 300 kBq/kg body weight. The present invention also relates to a PSMA binding ligand as described above or below, a complex as described above or below, or a pharmaceutical composition as described herein above, for use in diagnosis, preferably for diagnosing a cell proliferative disease or disorder, in particular prostate cancer and/or metastases thereof. Further, the present invention also relates to a PSMA binding ligand as described above or below a complex as described above or below, or a pharmaceutical composition as described above or below, for use in medicine, preferably for treating or preventing a cell proliferative disease or disorder, in particular prostate cancer and/or metastases thereof.

The term “diagnosing”, as used herein, refers to assessing whether a subject suffers from a disease or disorder, preferably cell proliferative disease or disorder, or not. As will be understood by those skilled in the art, such an assessment, although preferred to be, may usually not be correct for 100% of the investigated subjects. The term, however, requires that a, preferably statistically significant, portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, etc., Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, or 0.05. As will be understood by the skilled person, diagnosing may comprise further diagnostic assessments, such as visual and/or manual inspection, determination of tumor biomarker concentrations in a sample of the subject, X-ray examination, and the like. The term includes individual diagnosis of as well as continuous monitoring of a patient. Monitoring, i.e. diagnosing the presence or absence of cell proliferative disease or the symptoms accompanying it at various time points, includes monitoring of patients known to suffer from cell proliferative disease as well as monitoring of subjects known to be at risk of developing cell proliferative disease. Furthermore, monitoring can also be used to determine whether a patient is treated successfully or whether at least symptoms of cell proliferative disease can be ameliorated over time by a certain therapy. Moreover, the term also includes classifying a subject according to a usual classification scheme, e.g. the T1 to T4 staging, which is known to the skilled person.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, as specified herein above. The term “preventing” and “prevention” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the PSMA binding ligand according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed herein above.

Preferably, treatment and/or prevention comprises administration of at least one PSMA binding ligand and/or at least one complex as specified elsewhere herein, more preferably at an activity dosage and/or compound dosage as specified above.

The term “cell proliferative disease”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body (metastasis). Preferably, also included by the term cancer is a relapse. Thus, preferably, the cancer is a solid cancer, a metastasis, or a relapse thereof. Preferably, the cell proliferative disease is an uncontrolled proliferation of cells comprising cells expressing PSMA.

Thus, preferably, the cell proliferative disease is a PSMA expressing cancer. The term “PSMA expressing cancer” refers to any cancer whose cancerous cells express Prostate Specific Membrane Antigen (PSMA). Preferably cancers (or cancer cells) that may be treated according to the invention are selected among prostate cancer, conventional renal cell cancers, cancers of the transitional cells of the bladder, lung cancers, testicular-embryonal cancers, neuroendocrine cancers, colon cancers, brain tumors and breast cancers, more preferably are selected among PSMA-positive prostate cancer, PSMA-positive renal cell cancers, PSMA-positive cancers of the transitional cells of the bladder, PSMA-positive lung cancers, PSMA-positive testicular-embryonal cancers, PSMA-positive neuroendocrine cancers, PSMA-positive colon cancers, PSMA-positive brain tumors, and PSMA-positive breast cancers. Whether a cancer is PSMA-positive can be established by the skilled person by methods known in the art, e.g. in vitro by immunostaining of a cancer sample, or in vivo e.g. by PSMA scintigraphy, preferably both as described in Kratochwil et al. (2017, J Nucl Med 58 (10): 1624. In particularly preferred aspects of the invention, said PSMA expressing cancer is prostate cancer or breast cancer, more preferably prostate cancer; and even more preferably advanced-stage prostate cancer. Thus, preferably, the cell proliferative disease is prostate cancer stage T2, more preferably stage T3, most preferably stage T4. Preferably, the cell proliferative disease is metastatic prostate cancer, more preferably is metastatic castration-resistant prostate cancer. Advantageously, it has been shown in the studies underlying the present invention that administration of the PSMA binding ligand and/or complexes of the present invention to a patient results in an improved pharmacokinetic profile, in particular improved renal excretion with essentially unchanged enrichment in target tissue, preferably cell proliferative tissue, more preferably cancer tissue, as compared to e.g. the meanwhile commonly used PSMA-617. Due to the improved excretion, adverse side effects on non-target tissues, in particular the salivary and/or lacrimal glands, can be avoided and/or reduced. This is advantageous, because the adverse side effects on the salivary glands are considered as dosage-limiting (cf. Kratochwil et al. (2017, J Nucl Med 58 (10): 1624). Based on the finding of the present invention, larger amounts of compounds and/or complexes and in particular higher doses of radioactivity can be administered to a patient as compared to the compounds and complexes described in the art. Thus, the therapeutic window is broader than with the compounds presently in use. Also advantageously, the PSMA binding ligands of the present invention provide for improved diagnosis, since the co-labelling of irrelevant tissue and organs, in particular salivary glands, lacrimal glands and/or kidneys, is reduced.

Thus, the PSMA binding ligands and/or complexes of the present invention allow for the treatment of PSMA-expressing cancers, especially prostate cancer, and metastases thereof, and/or the diagnosis of PSMA-expressing cancers, especially prostate cancer, and metastases thereof, wherein the PSMA binding ligands and/or complexes display an advantageous renal excretion profile, preferably with a favorable clearance acceleration. Thus, adverse side effects on the patient's kidney are diminished. Thus, the present invention also relates to a PSMA binding ligands and/or complexes of the present invention or a pharmaceutical composition, as described above, for treating and/or preventing PSMA expressing cancer, in particular prostate cancer and/or metastases thereof, in a patient in need thereof, the subject suffering from renal failure.

As detailed herein above and in the Examples, the compounds as specified herein provide for accelerated excretion while maintaining essentially the same enrichment in target tissue as e.g. PSMA-617, so adverse effects on non-target tissues, preferably the salivary and/or lacrimal glands, are avoided or reduced. Thus, treatment and/or diagnosis as specified herein has less or less severe adverse side effects, e.g. on the salivary glands and/or lacrimal glands, or is preferably not accompanied by adverse side effects, in particular on the salivary glands and/or lacrimal glands.

Preferably, the PSMA binding ligands of the present invention allow for reduction and/or avoidance of adverse side effects, e.g. on the salivary glands and/or lacrimal glands, while maintaining therapeutic efficacy essentially unchanged. As will be understood by the skilled person in view of the above, the PSMA binding ligands as specified herein preferably further make a use of higher concentrations of the compounds and/or higher doses of radioactivity feasible while at least not increasing adverse effects, which may be particularly useful in diagnostic applications to detect e.g. small metastasis or small amounts of remaining tumor tissue, and/or in treatment.

Accordingly, the PSMA binding ligands and/or complexes of the present invention allow for the treatment of PSMA-expressing cancers, especially prostate cancer, and metastases thereof, and/or the diagnosis of PSMA-expressing cancers, especially prostate cancer, and metastases thereof, wherein xerostomia is avoided.

Preferably the PSMA binding ligand, as described above or below, or the complex, as described above or below, or the pharmaceutical composition, as described above or below, are used for in vivo imaging and radiotherapy. Suitable pharmaceutical compositions may contain a radio imaging agent, or a radiotherapeutic agent that has a radionuclide either as an element, i.e. radioactive iodine, or a radioactive metal chelate complex of the PSMA binding ligand in an amount sufficient for imaging, together with a pharmaceutically acceptable radiological vehicle. The radiological vehicle should be suitable for injection or aspiration, such as human serum albumin; aqueous buffer solutions, e.g., tris(hydromethyl)-aminomethane (and its salts), phosphate, citrate, bicarbonate, etc.; sterile water physiological saline; and balanced ionic solutions containing chloride and or dicarbonate salts or normal blood plasma cautions such as calcium potassium, sodium and magnesium.

The concentration of the imaging agent or the therapeutic agent in the radiological vehicle should be sufficient to provide satisfactory imaging. Appropriate dosages have been described herein above. The imaging agent or therapeutic agent should be administered so as to remain in the patient for about 1 hour to 10 days, although both longer and shorter time periods are acceptable. Therefore, convenient ampoules containing 1 to 10 mL of aqueous solution may be prepared.

Imaging may be carried out in a manner known to the skilled person, for example by injecting a sufficient amount of the imaging composition to provide adequate imaging and then scanning with a suitable imaging or scanning machine, such as a tomograph or gamma camera. In certain embodiments, a method of imaging a region in a patient includes the steps of: (i) administering to a patient a diagnostically effective amount of a PSMA binding ligand complexed with a radionuclide; (ii) exposing a region of the patient to the scanning device; and (ii) obtaining an image of the region of the patient. In certain embodiments of the region imaged is the head or thorax. In other embodiments, the PSMA binding ligandss and complexes target the PSMA protein. Thus, in some embodiments, a method of imaging tissue such as spleen tissue, kidney tissue, or PSMA-expressing tumor tissue is provided including contacting the tissue with a complex synthesized by contacting a radionuclide and PSMA binding ligand, as described above.

The amount of the PSMA binding ligand of the present invention, or a formulation comprising a complex of the PSMA binding ligand, or its salt, solvate, stereoisomer, or tautomer that is administered to a patient depends on several physiological factors. These factors are known by the physician, including the nature of imaging to be carried out, tissue to be targeted for imaging or therapy and the body weight and medical history of the patient to be imaged or treated using a radiopharmaceutical.

Accordingly in another aspect, the invention provides a method for treating a patient by administering to a patient a therapeutically effective amount of a complex, as described above or below, to treat a patient suffering from a cell proliferative disease or disorder. Specifically, the cell proliferative disease or disorder to be treated or imaged using a PSMA binding ligand, pharmaceutical composition or radiopharmaceutical in accordance with this invention is a cancer, for example, prostate cancer and/or prostate cancer metastasis in e.g. lung, liver, kidney, bones, brain, spinal cord, bladder, etc.

The PSMA binding ligands of the invention may e.g. be synthesized in solution as well as on solid phase using e.g. standard peptide coupling procedures, such as Fmoc solid phase coupling procedures. Preferably, the chelator is coupled to the remaining part of the molecule in the last coupling step followed by a deprotection step and in case of solid phase chemistry, cleavage from the resin. However, other synthetic procedures are possible and known to the skilled person. A preferred synthesis of the PSMA binding ligands of the present invention is described in detail in the example section

Summarizing the findings of the present invention, the following embodiments are preferred:

1. PSMA binding ligand or a pharmaceutically acceptable salt or solvate thereof comprising a PSMA binding motif Q and a chelator residue A linked via at least one linker L^{AQ} comprising at least one, optionally N-alkylated, neutral amino acid X₁, wherein X₁ is preferably selected from the group consisting of, optionally N-alkylated, Gly, Ala, βAla, Phe, amino hexanoic acid (Ahx=—NH—(CH₂)₅—C(═O)—) and amino acids comprising at least one —(CH₂—CH₂—O)— group between the N-terminus and the C-terminus, more preferably, wherein X₁ is selected from the group consisting of Gly, Ala, βAla, Phe and amino acid residues having the structure —NH—CH₂—CH₂—O—(CH₂—CH₂—O)ₓ₁—(CH₂)_{y1}—C(═O)— with x1 being an integer in the range of from 0 to 15, and y1 being 1 or 2, or wherein X₁ is Tyr

2. PSMA binding ligand according to embodiment 1 or a pharmaceutically acceptable salt or solvate thereof, the PSMA binding ligand having the structure (I)

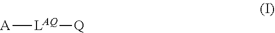

3. PSMA binding ligand according to embodiment 1 or 2 or a pharmaceutically acceptable salt or solvate thereof, the PSMA binding motif Q having the structure

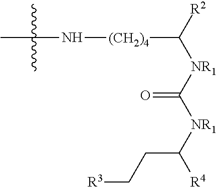

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂.

4. PSMA binding ligand according to any one of embodiments 1 to 3 or a pharmaceutically acceptable salt or solvate thereof,
wherein A is a chelator residue derived from a chelator selected from the group consisting of 1,4,7,10-tetraazacyclododecane-N,N′,N″,N′″-tetraacetic acid (=DOTA), N,N″-bis [2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl (2-carboxyethyl)phosphinic acid]-4,7-bis [methyl (2-hydroxymethyl)phosphinic acid] (NOPO), 3,6,9,15-tetraazabicyclo [9.3.1]pentadeca-1 (15), 11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA).

5. The PSMA binding ligand according to any one of embodiments 1 to 4 or a pharmaceutically acceptable salt or solvate thereof, wherein A is a chelator residue having a structure selected from the group consisting of

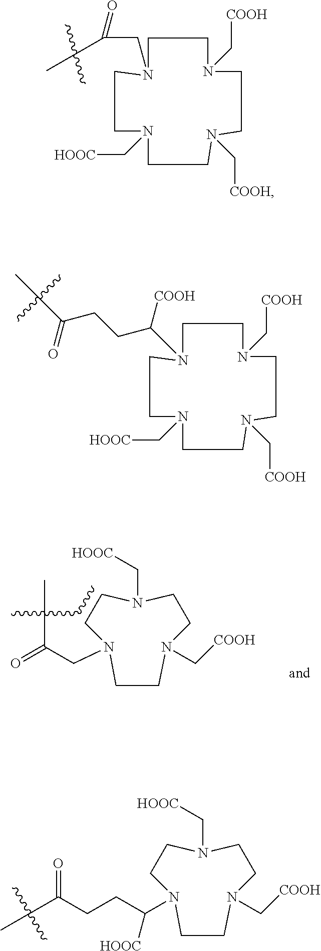

6. The PSMA binding ligand according to any one of embodiments 1 to 5 or a pharmaceutically acceptable salt or solvate thereof, wherein A is a chelator residue having the structure

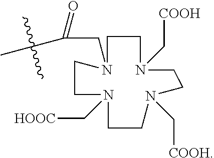 [SMILES: *C(=O)CN1CCN(CC(=O)O)CCN(CC(=O)O)CCN(CC(=O)O)CC1]

7. PSMA binding ligand according to any one of embodiments 1 to 6 or a pharmaceutically acceptable salt or solvate thereof, wherein the linker L^{AQ} comprises at least one amino acid building block AS^{a}, wherein AS^{a} has the structure

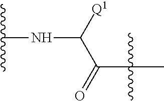

wherein Q¹ is selected from the group consisting of alkylaryl, arylalkyl, aryl, alkylheteroaryl, heteroarylalkyl and heteroaryl.

8. PSMA binding ligand according to any one of embodiments 1 to 7 or a pharmaceutically acceptable salt or solvate thereof, wherein the linker L^{AQ} comprises at least one amino acid building block AS^{b}, wherein AS^{b} has the structure (b)

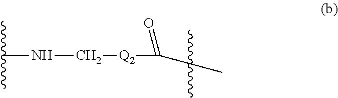

wherein Q² is selected from the group consisting of aryl, alkylaryl, arylalkyl, cycloalkyl, heterocycloalkyl, heteroaryl, heteroarylalkyl and alkylheteroaryl, preferably wherein Q² is

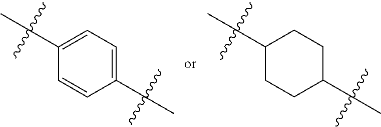 [SMILES: *c1ccc(*)cc1 ; *C1CCC(*)CC1]

more preferably

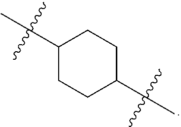 [SMILES: *C1CCC(*)CC1]

9. PSMA binding ligand according to any one of embodiments 1 to 6 or a pharmaceutically acceptable salt or solvate thereof, wherein the linker L^{AQ} comprises at least one amino acid building block AS^{a} and at least one amino acid building block AS^{b}, wherein AS^{a} has the structure

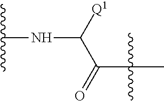

wherein Q¹ is selected from the group consisting of alkylaryl, arylalkyl, aryl, alkylheteroaryl, heteroarylalkyl and heteroaryl, and wherein AS^{b} has the structure (b)

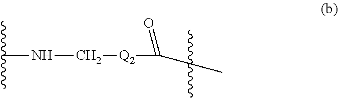

wherein Q² is selected from the group consisting of aryl, alkylaryl, arylalkyl, cycloalkyl, heterocycloalkyl, heteroaryl, heteroarylalkyl and alkylheteroaryl, preferably wherein Q² is

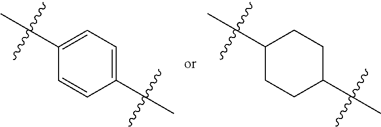 [SMILES: *c1ccc(*)cc1 ; *C1CCC(*)CC1]

more preferably

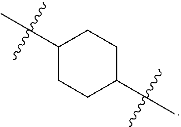 [SMILES: *C1CCC(*)CC1]

10. PSMA binding ligand according any one of embodiments 1 to 8 or a pharmaceutically acceptable salt or solvate thereof, the PSMA binding ligand having the structure (Ia)

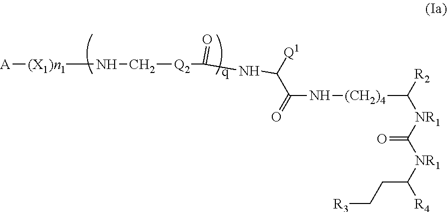

wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂,
Q¹ is selected from the group consisting of alkylaryl, arylalkyl, aryl, alkylheteroaryl, heteroarylalkyl and heteroaryl,
Q² is selected from the group consisting of aryl, alkylaryl, arylalkyl, cycloalkyl, heterocycloalkyl, heteroaryl, heteroarylalkyl and alkylheteroaryl,
and wherein q is an integer of from 0-3, and wherein n1 is an integer in the range of from 1 to 25.

11. The PSMA binding ligand according to embodiment 10 or a pharmaceutically acceptable salt or solvate thereof, wherein Q¹ preferably comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

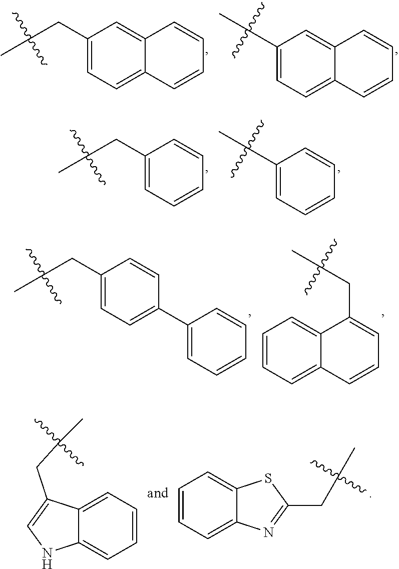

preferably wherein Q¹ is

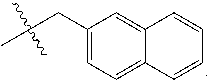 [SMILES: *Cc1ccc2ccccc2c1]

12. The PSMA binding ligand of any of one of embodiments 10 or 11, wherein R³, R² and R⁴ are —CO₂H and R¹ is H.

13. The PSMA binding ligand of any one of embodiments 10 to 12, wherein Q² is

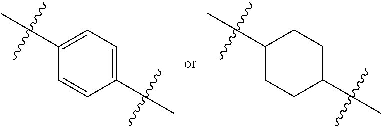 [SMILES: *c1ccc(*)cc1 ; *C1CCC(*)CC1]

preferably

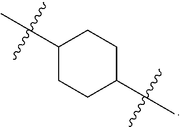 [SMILES: *C1CCC(*)CC1]

14. The PSMA binding ligand of any one of embodiments 1 to 13, the ligand having the structure

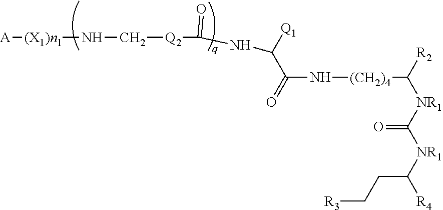

wherein A is a chelator residue having the structure

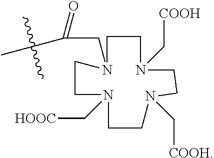 [SMILES: *C(=O)CN1CCN(CC(=O)O)CCN(CC(=O)O)CCN(CC(=O)O)CC1]

Q² is

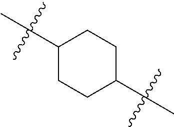 [SMILES: *C1CCC(*)CC1]

more preferably

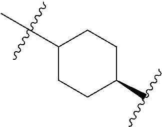

q is 1
Q¹ is

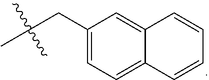 [SMILES: *Cc1ccc2ccccc2c1]

and wherein R³, R² and R⁴ are —CO₂H and R¹ is H and wherein n1 is an integer in the range of from 1 to 25.

15. The PSMA binding ligand of any one of embodiments 1 to 14, wherein X₁ is selected from the group consisting of Gly, Ala, βAla, Phe, —NH—(CH₂)₅—C(═O)— and amino acids comprising at least one —(CH₂—CH₂—O)— group between the N-terminus and the C-terminus.

16. The PSMA binding ligand of any one of embodiments 1 to 14, wherein X₁ is selected from the group consisting of Gly, Ala, βAla, Phe, —NH—(CH₂)₅—C(═O)— and amino acid residues having the structure —NH—CH₂—CH₂—O—(CH₂—CH₂—O)ₓ₁—(CH₂)_{y1}—C(═O)— with x1 being an integer in the range of from 0 to 15, and y1 being 1 or 2.

17. The PSMA binding ligand of any one of embodiments 1 to 16, wherein X₁ is selected from the group consisting of Gly, Ala, βAla and Phe.

18. The PSMA binding ligand of any one of embodiment 1 or 14, wherein the linker L^{AQ} comprises the building block (X₁)ₙ₁, wherein X₁ is selected from the group consisting of Gly, Ala, βAla and Phe or wherein X₁ is Tyr, and n1 is in the range of from 2 to 20, preferably 3 to 15, more preferably, 3, 5, 10 or 15.

19. The PSMA binding ligand of any one of embodiment 18, wherein n1 is 3.

20. The PSMA binding ligand of any one of embodiment 18, wherein n1 is 5.

21. The PSMA binding ligand of any one of embodiment 18, wherein n1 is 10.

22. The PSMA binding ligand of any one of embodiment 18, wherein n1 is 15.

23. The PSMA binding ligand of any one of embodiments 18 to 22, wherein X₁ is Gly.

24. The PSMA binding ligand of any one of embodiments 18 to 22, wherein X₁ is Ala.

25. The PSMA binding ligand of any one of embodiments 18 to 22, wherein X₁ is βAla.

26. The PSMA binding ligand of of any one of embodiments 18 to 22, wherein X₁ is Phe.

27. The PSMA binding ligand of embodiment 1 or 16, wherein the linker L^{AQ} comprises the building block (X₁)ₙ₁, with X₁ having the structure —NH—CH₂—CH₂—O—(CH₂—CH₂—O)ₓ₁—(CH₂)_{y1}—C(═O)— and with integer x1 being in the range of from 0 to 15, and y1 being 1 or 2 and with n1 being 1.

28. The PSMA binding ligand of embodiment 27, with integer x1 being 1, 3, 5 or 7.

29. The PSMA binding ligand of embodiment 27, with integer x1 being 1 and y1 being 1.

30. The PSMA binding ligand of embodiment 27, with integer x1 being 3 and y1 being 2.

31. The PSMA binding ligand of embodiment 27, with integer x1 being 5 and y1 being 2.

32. The PSMA binding ligand of embodiment 27, with integer x1 being 7 and y1 being 2.

33. The PSMA binding ligand of any one of embodiment 1 or 14, wherein the linker L^{AQ} comprises the building block (X₁)ₙ₁, wherein X₁ is-NH—(CH₂)₅—C(═O)— and n1 is in the range of from 2 to 20, preferably 1 to 4.

34. The PSMA binding ligand of any one of embodiment 33, wherein n1 is 1.

35. The PSMA binding ligand of any one of embodiment 33, wherein n1 is 2.

36. The PSMA binding ligand of any one of embodiment 33, wherein n1 is 3.

37. The PSMA binding ligand of any one of embodiment 33, wherein n1 is 3.

38. The PSMA binding ligand of any one of embodiments 1 to 37 the ligand having the structure as shown in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, FIG. 18, FIG. 19, FIG. 20, FIG. 21 or FIG. 22.

39. Complex comprising
(a) a radionuclide, and
(b) the PSMA binding ligand of any one of embodiments 1 to 37 or a pharmaceutically acceptable salt or solvate thereof.

40. The complex of embodiment 39, wherein, the radionuclide is selected from the group consisting ⁸⁹Zr, ⁴⁴Sc, ¹¹¹In, ⁹⁰Y, ⁶⁶Ga, ⁶⁷Ga, ⁶⁸Ga, ¹⁷⁷Lu, ⁹⁹Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe, and radionuclides of Pb (such as ²⁰³Pb and ²¹²Pb, ²¹¹Pb, ²¹³Pb, ²¹⁴Pb, ²⁰⁹Pb, ²²⁵Ac, ¹⁹⁸Pb, ¹⁹⁷Pb), more preferably selected from the group consisting ⁹Y, ⁶⁸Ga, ¹⁷⁷Lu, ²²⁵Ac, and ²¹³Bi, more preferably, the radionuclide is ¹⁷⁷Lu or ²²⁵Ac.

41. A pharmaceutical composition comprising a PSMA binding ligand of any one of embodiments 1 to 38 or a complex of embodiment 39 or 40.

42. A PSMA binding ligand of any one of embodiments 1 to 38 or a complex of embodiment 39 or 40 or a pharmaceutical composition of embodiment 41 for use in medicine, preferably for treating and/or preventing PSMA expressing cancer, in particular prostate cancer and/or metastases thereof.

43. The PSMA binding ligand of any one of embodiments 1 to 38 or a complex of embodiment 39 or 40 or a pharmaceutical composition of embodiment 41 for use in diagnostics.

44. The PSMA binding ligand of any one of embodiments 1 to 38 or a complex of embodiment 39 or 40 or a pharmaceutical composition of embodiment 41 for use in the diagnosis of cancer, preferably of PSMA expressing cancer, in particular of prostate cancer and/or metastases thereof.

45. The PSMA binding ligand, the complex, or the pharmaceutical composition for use of embodiment 43 or 44, wherein the radionuclide is a β-emitter, more preferably 177Lu and wherein preferably the activity dosage of the complex is at least 100 kBq/kg body weight, more preferably at least 500 kBq/kg body weight, most preferably at least 1 MBq/kg body weight.

46. The PSMA binding ligand, complex, or pharmaceutical composition for use of embodiment 42, wherein the radionuclide is an α-emitter, more preferably is 225Ac and wherein preferably the activity dosage of the complex is at least 75 kBq/kg body, more preferably at least 100 kBq/kg body, weight.

47. The PSMA binding ligand of any one of embodiments 18 to 22, wherein X₁ is Tyr.

48. The PSMA binding ligand of any one of claims 1 to 8, wherein the linker L^{AQ} comprises the building block (X₁)ₙ₁, and wherein
X₁ is selected from the group consisting of Gly, Ala, βAla and Phe and n1 is in the range of from 2 to 20, preferably 3 to 15, more preferably, 3, 5, 10 or 15 or
X₁ has the structure —NH—CH₂—CH₂—O—(CH₂—CH₂—O)ₓ₁—(CH₂)_{y1}—C(═O)— and with integer x1 being in the range of from 0 to 15, and y1 being 1 or 2 and with n1 being 1, or
X₁ has the structure —NH—(CH₂)₅—C(═O)— and n1 is in the range of from 1 to 20, preferably 1 to 4, or
X1 is Tyr and n1 is in the range of from 2 to 20, preferably 3 to 15, more preferably, 3, 5, 10 or 15.

All references cited throughout this specification are herewith incorporated by reference with respect to the specifically mentioned disclosure content as well as in their entireties.

### FIGURES

FIG. 1: Structure of DOTA-Gly₃-Chx-2-NaI-Lys-urea-Glu

FIG. 2: Structure of DOTA-Gly₅-Chx-2-NaI-Lys-urea-Glu

FIG. 3: Structure of DOTA-Gly₁₀-Chx-2-NaI-Lys-urea-Glu

FIG. 4: Structure of DOTAGly₁₅-Chx-2-NaI-Lys-urea-Glu

FIG. 5: Structure of DOTA-Ala₃-Chx-2-NaI-Lys-urea-Glu

FIG. 6: Structure of DOTA-Ala₅-Chx-2-NaI-Lys-urea-Glu

FIG. 7: Structure of DOTA-Ala₁₀-Chx-2-NaI-Lys-urea-Glu

FIG. 8: Structure of DOTA-Ala₁₅-Chx-2-NaI-Lys-urea-Glu

FIG. 9: Structure of DOTA-βAla₃-Chx-2-NaI-Lys-urea-Glu

FIG. 10: Structure of DOTA-βAla₅-Chx-2-NaI-Lys-urea-Glu

FIG. 11: Structure of DOTA-βAla₁₀-Chx-2-NaI-Lys-urea-Glu

FIG. 12: Structure of DOTA-βAla₁₅-Chx-2-NaI-Lys-urea-Glu

FIG. 13: Structure of DOTA-Phe₃-Chx-2-NaI-Lys-urea-Glu

FIG. 14: Structure of DOTA-Phe₅-Chx-2-NaI-Lys-urea-Glu

FIG. 15: Structure of DOTA-PEG₂-Chx-2-NaI-Lys-urea-Glu

FIG. 16: Structure of DOTA-PEG₄-Chx-2-NaI-Lys-urea-Glu

FIG. 17: Structure of DOTA-PEG₆-Chx-2-NaI-Lys-urea-Glu

FIG. 18: Structure of DOTA-PEG₈-Chx-2-NaI-Lys-urea-Glu

FIG. 19: Structure of DOTA-Ahx₁-Chx-2-NaI-Lys-urea-Glu

FIG. 20: Structure of DOTA-Ahx₂-Chx-2-NaI-Lys-urea-Glu

FIG. 21: Structure of DOTA-Ahx₃-Chx-2-NaI-Lys-urea-Glu

FIG. 22: Structure of DOTA-Ahx₄-Chx-2-NaI-Lys-urea-Glu

FIG. 23: Structure of DOTA-Tyr₃-Chx-2-NaI-Lys-urea-Glu

FIG. 24: Structure of DOTA-Tyr₅-Chx-2-NaI-Lys-urea-Glu

FIG. 25: Cell surface binding and internalization in percent of applied radioactivity of the ¹⁷⁷Lu-labeled compounds without blocking or with blocking after 45 min (n=3).

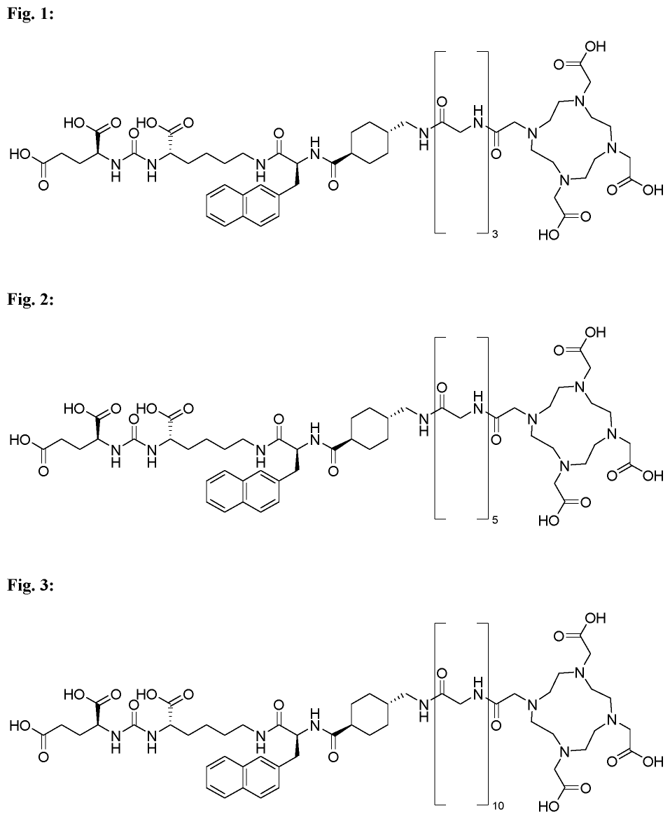

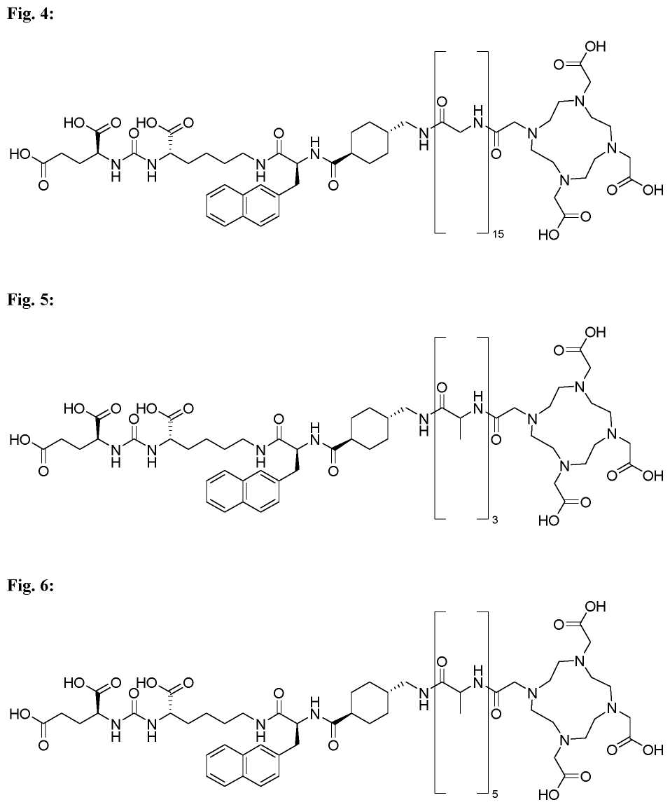

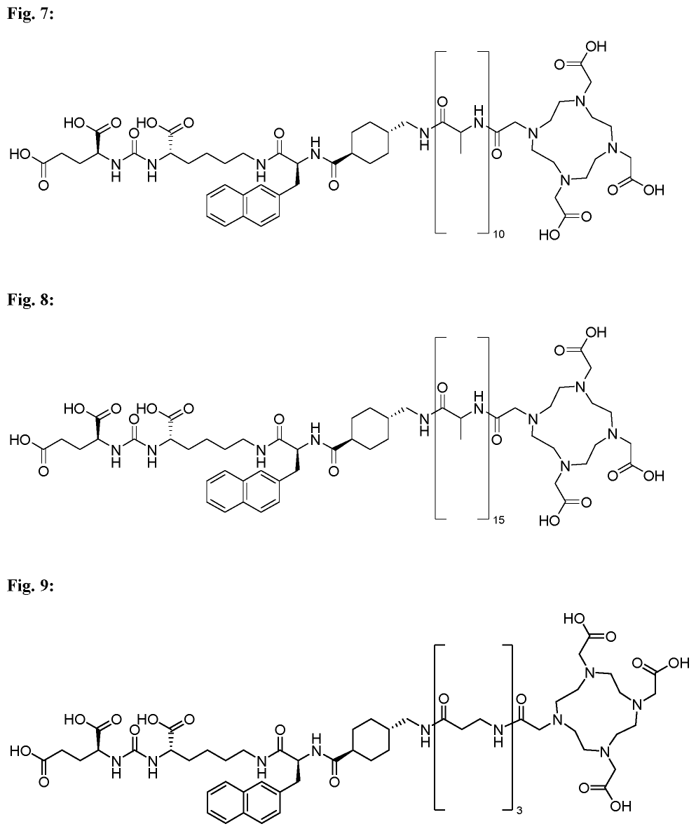

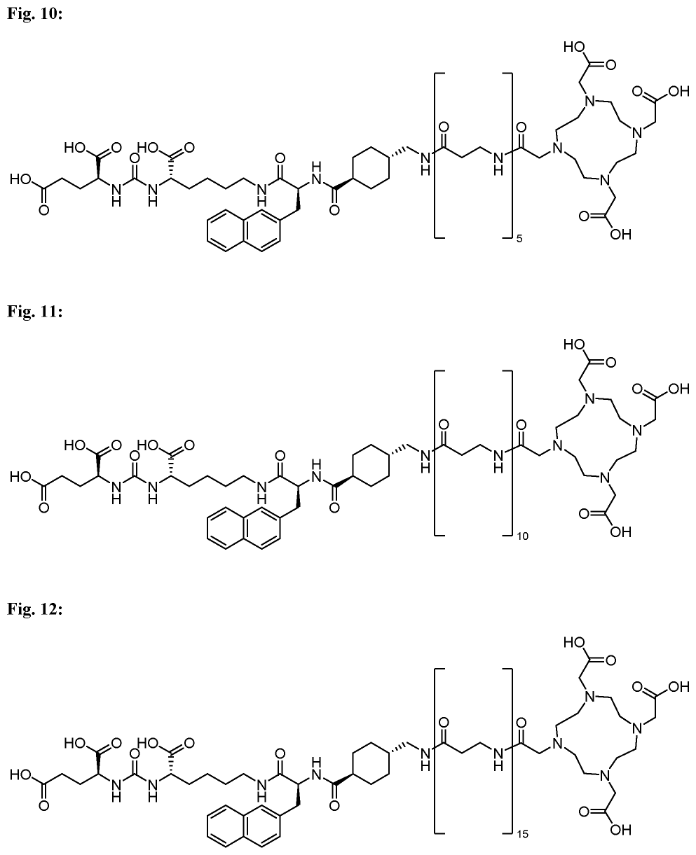

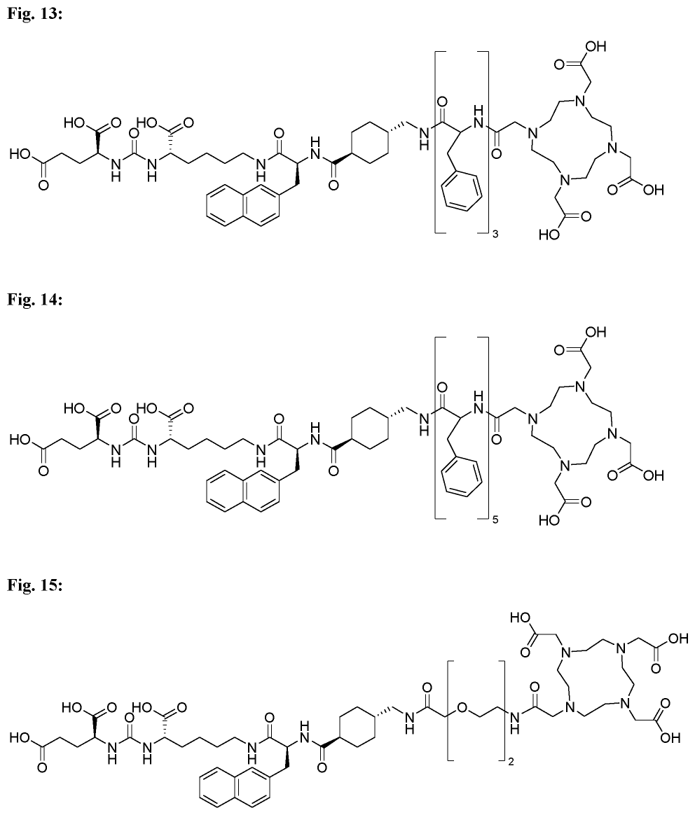

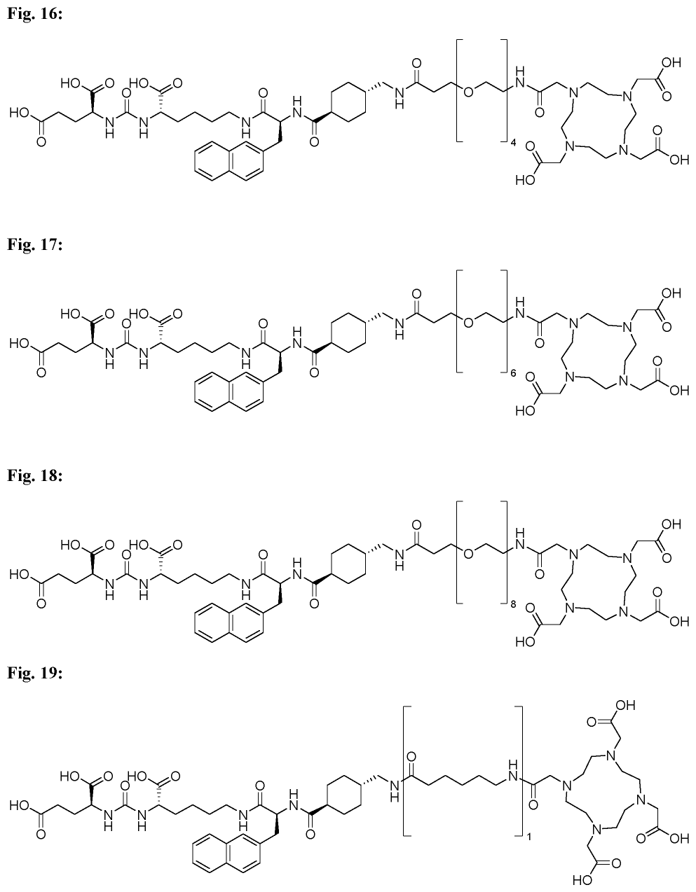

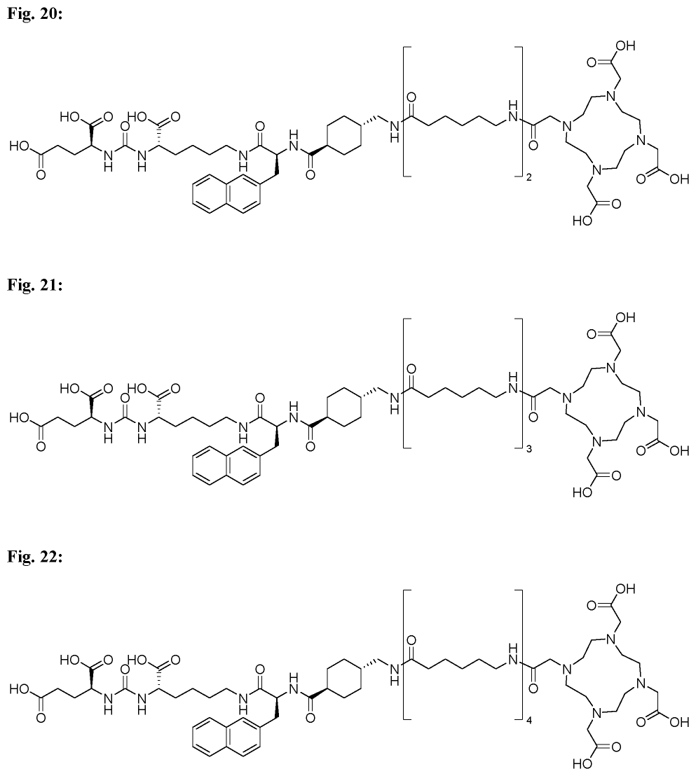

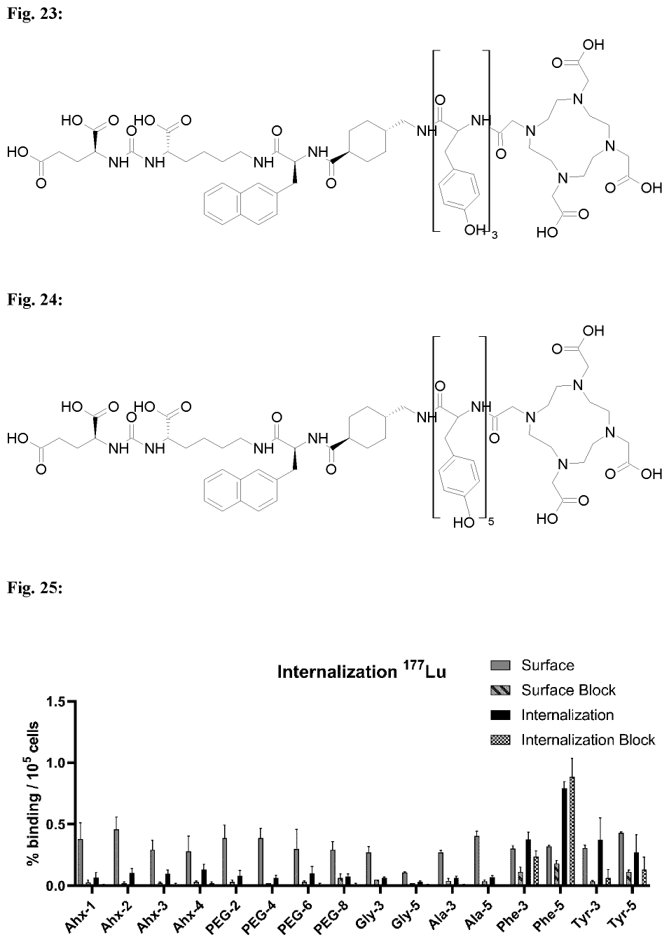

### EXAMPLES

All commercially available chemicals were of analytical grade and used without further purification. ⁶⁸Ga (half-life 68 min) was obtained from a ⁶⁸Ge/⁶⁸Ga generator (Galliapharm® Ge-68/Ga-68 Generator, Eckert&Ziegler) and 177Lu (half-life 6.6 d) was purchased from ITG. The compounds were purified using semipreparative reversed-phase high performance liquid chromatography (RP-HPLC; Chromolith Semi Prep RP-18e, 100×10 mm; Merck, Darmstadt, Germany). Compound analysis was performed using analytical RP-HPLC (RP-HPLC; Chromolith RP-18e, 100×4.6 mm; Merck, Darmstadt, Germany). Analytical HPLC runs were performed using a linear gradient (5% A (0.1% aqueous TFA) to 100% B (0.1% TFA in CH₃CN)) in 10 min at 2 mL/min. The system Agilent Technologies 1200 Series was equipped with a variable UV and a gamma detector (Ramona*, Elysia). UV absorbance was measured at 220 and 280 nm, respectively. For mass spectrometry a MALDI-MS (Daltonics Microflex, Bruker Daltonics, Bremen, Germany) was used.

Synthesis of DOTA-Glyₓ-Chx-2-NaI-PSMA, DOTA-Alaₓ-Chx-2-NaI-PSMA, DOTA-βAlaₓ-Chx-2-NaI-PSMA (x=3, 5, 10, 15)

### (“PSMA”=“Lys-urea-Glu”)

The synthesis of the pharmacophore Glu-urea-Lys was performed as described previously (1). Briefly, the synthesis started with the formation of the isocyanate of the glutamyl moiety using triphosgene. A resin-immobilized (2-chloro-tritylresin, Merck, Darmstadt) ¿-allyloxycarbonyl protected lysine was added and reacted for 16 h with gentle agitation. The resin was filtered off and the allyloxy-protecting group was removed by reacting twice with Pd(PPh₃)₄ (0.3 eq.) and morpholine (15 eq.) under ambient conditions (1 h, RT).

Subsequently, the linker between the PSMA pharmacophore and the chelator was introduced by standard Fmoc solid phase protocol. In a first step Fmoc-2-NaI—OH and N-Fmoc-tranexamic acid (5 eq. each) with HATU (5 eq.) and DIPEA (10 eq.) were coupled in DMF. Each coupling step was repeated one time. Depending on the amino acid sequence Fmoc-Gly-OH/Fmoc-Ala-OH or Fmoc-β-Ala-OH (5 eq./coupling) was coupled three, five, ten and fifteen-times, respectively, with HATU (5 eq.) and DIPEA (10 eq.) in DMF. Each coupling step was repeated one time.

Afterwards, tris(tBu)DOTA (tris(tBu)-ester of 1,4,7,10-tetraazacyclododecan-1,4,7,10-tetraacetic acid) (4 eq.) with HATU (4 eq.) and DIPEA (10 eq.) were coupled in DMF. The products were cleaved from the resin for 3 hours at RT using TFA/TIPS/H₂O (95/2.5/2.5, v/v/v) and identified with mass spectrometry.

TABLE 2
Analytical data of the final compounds. Mass spectrometry
(MALDI-MS) was performed with the metal-free substances.

| | m/z | m/z |
|---|---|---|
| | (g/mol, M_{calc.}) | (M_{found.}) |
| DOTA-Gly₃-Chx-2-NaI-Lys-urea-Glu | 1212 | 1210 |
| DOTA-Gly₅-Chx-2-NaI-Lys-urea-Glu | 1326 | 1326 |
| DOTA-Gly₁₀-Chx-2-NaI-Lys-urea-Glu | 1612 | 1612 |
| DOTA-Gly₁₅-Chx-2-NaI-Lys-urea-Glu | 1897 | 1898 |
| DOTA-Ala₃-Chx-2-NaI-Lys-urea-Glu | 1255 | 1255 |
| DOTA-Ala₅-Chx-2-NaI-Lys-urea-Glu | 1397 | 1397 |
| DOTA-Ala₁₀-Chx-2-NaI-Lys-urea-Glu | 1752 | 1752 |
| DOTA-Ala₁₅-Chx-2-NaI-Lys-urea-Glu | 2107 | In process |
| DOTA-βAla₃-Chx-2-NaI-Lys-urea-Glu | 1255 | 1254 |
| DOTA-βAla₅-Chx-2-NaI-Lys-urea-Glu | 1397 | 1396 |
| DOTA-βAla₁₀-Chx-2-NaI-Lys-urea-Glu | 1752 | 1752 |
| DOTA-βAla₁₅-Chx-2-NaI-Lys-urea-Glu | 2107 | 2107 |

Synthesis of DOTA-Phe_{y}-Chx-2-NaI-Lys-urea-Glu (y=3, 5) and DOTA-Tyr_{y}-Chx-2-NaI-Lys-urea-Glu (y=3, 5)

The synthesis of the pharmacophore Glu-urea-Lys was performed as described previously (1). Briefly, the synthesis started with the formation of the isocyanate of the glutamyl moiety using triphosgene. A resin-immobilized (2-chloro-tritylresin, Merck, Darmstadt) ε-allyloxycarbonyl protected lysine was added and reacted for 16 h with gentle agitation. The resin was filtered off and the allyloxy-protecting group was removed by reacting twice with Pd(PPh₃)₄ (0.3 eq.) and morpholine (15 eq.) under ambient conditions (1 h, RT).

Subsequently, the linker between the PSMA pharmacophore and the chelator was introduced by standard Fmoc solid phase protocol. In a first step Fmoc-2-NaI—OH and N-Fmoc-tranexamic acid (5 eq. each) with HATU (5 eq.) and DIPEA (10 eq.) were coupled in DMF. Each coupling step was repeated one time. Depending on the amino acid sequence Fmoc-Phe-OH (5 eq./coupling) or Fmoc-Tyr(tBu)-OH was coupled three or five-times, respectively, with HATU (5 eq.) and DIPEA (10 eq.) in DMF. Each coupling step was repeated one time.

Afterwards, tris((Bu)DOTA (tris(tBu)-ester of 1,4,7,10-tetraazacyclododecan-1,4,7,10-tetraacetic acid) (4 eq.) with HATU (4 eq.) and DIPEA (10 eq.) were coupled in DMF. The products were cleaved from the resin for 3 hours at RT using TFA/TIPS/H₂O (95/2.5/2.5, v/v/v) and identified with mass spectrometry.

TABLE 3
Analytical data of the final compounds. Mass spectrometry
(MALDI-MS) was performed with the metal-free substances.

| | m/z | m/z |
|---|---|---|
| | (g/mol, M_{calc.}) | (M_{found.}) |
| DOTA-Phe₃-Chx-2-NaI-Lys-urea-Glu | 1483 | 1484 |
| DOTA-Phe₅-Chx-2-NaI-Lys-urea-Glu | 1777 | 1778 |
| DOTA-Tyr₃-Chx-2-NaI-Lys-urea-Glu | 1531 | 1531 |
| DOTA-Tyr₅-Chx-2-NaI-Lys-urea-Glu | 1857 | 1857 |

Synthesis of PSMA-NaI-Chx-Ahx_{z}-DOTA (z=1, 2, 3, 4)

The synthesis of the pharmacophore Glu-urea-Lys was performed as described previously (1). Briefly, the synthesis started with the formation of the isocyanate of the glutamyl moiety using triphosgene. A resin-immobilized (2-chloro-tritylresin, Merck, Darmstadt) &-allyloxycarbonyl protected lysine was added and reacted for 16 h with gentle agitation. The resin was filtered off and the allyloxy-protecting group was removed by reacting twice with Pd(PPh₃)₄ (0.3 eq.) and morpholine (15 eq.) under ambient conditions (1 h, RT).

Subsequently, the linker between the PSMA pharmacophore and the chelator was introduced by standard Fmoc solid phase protocol. In a first step Fmoc-2-NaI—OH and N-Fmoc-tranexamic acid (5 eq. each) with HATU (5 eq.) and DIPEA (10 eq.) were coupled in DMF. Each coupling step was repeated one time. Depending on the amino acid sequence 6-(Fmoc-amino)-hexanoic acid (5 eq./coupling) was coupled one, two, three or four-times, respectively, with HATU (5 eq.) and DIPEA (10 eq.) in DMF. Each coupling step was repeated one time.

Afterwards, tris(tBu)DOTA (tris((Bu)-ester of 1,4,7,10-tetraazacyclododecan-1,4,7,10-tetraacetic acid) (4 eq.) with HATU (4 eq.) and DIPEA (10 eq.) were coupled in DMF. The products were cleaved from the resin for 3 hours at RT using TFA/TIPS/H₂O (95/2.5/2.5, v/v/v) and identified with mass spectrometry.

TABLE 4
Analytical data of the final compounds. Mass spectrometry
(MALDI-MS) was performed with the metal-free substances.

| | m/z | m/z |
|---|---|---|
| | (g/mol, M_{calc.}) | (M_{found.}) |
| DOTA-Ahx₁-Chx-2-NaI-Lys-urea-Glu | 1154 | 1154 |
| DOTA- Ahx₂-Chx-2-NaI-Lys-urea-Glu | 1267 | 1267 |
| DOTA- Ahx₃-Chx-2-NaI-Lys-urea-Glu | 1381 | 1380 |
| DOTA- Ahx₄-Chx-2-NaI-Lys-urea-Glu | 1494 | 1495 |

Ahx = Amino hexanoic acid

### Synthesis of DOTA-PEG2-Chx-2-NaI-Lys-urea-Glu, DOTA-PEG4-Chx-2-NaI-Lys-urea-Glu, DOTA-PEG6-Chx-2-NaI-Lys-urea-Glu, DOTA-PEG8-Chx-2-NaI-Lys-urea-Glu,

The synthesis of the pharmacophore Glu-urea-Lys was performed as described previously (1). Briefly, the synthesis started with the formation of the isocyanate of the glutamyl moiety using triphosgene. A resin-immobilized (2-chloro-tritylresin, Merck, Darmstadt) &-allyloxycarbonyl protected lysine was added and reacted for 16 h with gentle agitation. The resin was filtered off and the allyloxy-protecting group was removed by reacting twice with Pd(PPh₃)₄ (0.3 eq.) and morpholine (15 eq.) under ambient conditions (1 h, RT).

Subsequently, the linker between the PSMA pharmacophore and the chelator was introduced by standard Fmoc solid phase protocol. In a first step Fmoc-2-NaI—OH and N-Fmoc-tranexamic acid (4 eq. each) with HATU (4 eq.) and DIPEA (10 eq.) were coupled in DMF.

Depending on the amino acid sequence Fmoc-8-amino-3,6-dioxaoctanoic acid (5 eq.) or Fmoc-15-amino-4,7,10,13-tetraoxapentadecanoic acid (5 eq.) or Fmoc-21-amino-4,7,10,13,16,19-hexaoxaheneicosanoic acid (5 eq.) or Fmoc-27-amino-4,7,10,13,16,19-22-25-octaoxaheptacosanoic acid (5 eq.) was coupled, respectively, with HATU (5 eq.) and DIPEA (10 eq.) in DMF. Each coupling step was repeated once for every peptide with the individual PEG-reactant.

Afterwards, tris((Bu)DOTA (tris((Bu)-ester of 1,4,7,10-tetraazacyclododecan-1,4,7,10-tetraacetic acid) (3 eq.) with HATU (3 eq.) and DIPEA (10 eq.) were coupled in DMF. DOTA-coupling was also repeated once for every peptide. The products were cleaved from the resin for 3 hours at RT using TFA/TIPS/H₂O (95/2.5/2.5, v/v/v) and identified with mass spectrometry.

TABLE 5
Analytical data of the final compounds. Mass spectrometry
(MALDI-MS) was performed with the metal-free substances.

| | m/z | m/z |
|---|---|---|
| | (g/mol, M_{calc.}) | (M_{found.}) |
| DOTA-PEG₂-Chx-2-NaI-Lys-urea-Glu | 1186 | 1187 |
| DOTA-PEG₄-Chx-2-NaI-Lys-urea-Glu | 1289 | 1289 |
| DOTA-PEG₆-Chx-2-NaI-Lys-urea-Glu | 1378 | 1377 |
| DOTA-PEG₈-Chx-2-NaI-Lys-urea-Glu | 1466 | 1468 |

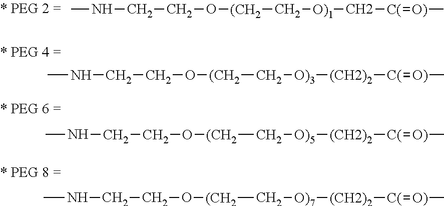

### In Vitro Characterization

### ¹⁷⁷Lu-Labeling

The precursor peptide [2 nmol in HEPES buffer (0.1 M, pH 7, 50 μL)] was added to 10 μL [¹⁷⁷Lu]LuCl₃ (˜10-30 MBq, 0.04 M HCl). The reaction mixture was incubated at 95° C. for 15 minutes. The radiochemical yield (RCY) was determined by RP-HPLC.

### Cell Culture

PSMA⁺ LNCaP cells (CRL-1740; ATCC; PSMA-positive) were cultured in RPMI medium supplemented with 10% fetal calf serum and 2 mmol/L L-glutamine (all from PAA). Cells were grown at 37° C. in humidified air with 5% CO₂ and were harvested using trypsin-ethylenediaminetetraacetic acid (trypsin-EDTA; 0.25% trypsin, 0.02% EDTA, Invitrogen).

### Cell Binding and Internalization

The internalization experiments were performed as described previously (2).

For internalization experiments, 10⁵ cells per well were seeded in poly-L-lysine coated 24-well cell culture plates 24 h before incubation. After washing, the cells were incubated with 30 nM of the radiolabeled DOTA-compound for 45 min at 37° C. (labeling was performed with 5 nmol of precursor peptide). Specific cell uptake was determined by blockage using 500 μM 2-PMPA (2-(Phosphonomethyl)pentanedioic acid). Cellular uptake was terminated by washing 3 times with 1 mL of ice-cold PBS. To remove surface-bound radioactivity, cells were incubated twice with 0.5 mL glycine-HCl in PBS (50 mM, pH=2.8) for 5 min. The cells were washed with 1 mL of ice-cold PBS and lysed using 0.3 N NaOH (0.5 mL). The surface-bound and the internalized fractions were measured in a gamma counter. The cell uptake was calculated as percent of the initially added radioactivity bound to 10⁵ cells [% ID/10⁵ cells].

TABLE A
Cell surface binding and internalization (without or with
blocking by 2-PMPA) of the ¹⁷⁷Lu-labeled compounds (7.5 pmol
precursor per 10⁵ cells) after 45 min (n = 3).

| | | Surface | | Internalization |
|---|---|---|---|---|
| | Surface* | Block* | Internalization* | Block* |
| | [% AR/10⁵ | [% AR/10⁵ | [% AR/10⁵ | [% AR/10⁵ |
| | cells] | cells] | cells] | cells] |
| Ahx-1 | 0.38 ± 0.14 | 0.03 ± 0.03 | 0.07 ± 0.05 | 0.01 ± 0.01 |
| Ahx-2 | 0.46 ± 0.11 | 0.02 ± 0.01 | 0.11 ± 0.04 | 0.01 ± 0.01 |
| Ahx-3 | 0.30 ± 0.08 | 0.03 ± 0.01 | 0.10 ± 0.04 | 0.02 ± 0.01 |
| Ahx-4 | 0.28 ± 0.13 | 0.03 ± 0.01 | 0.13 ± 0.05 | 0.02 ± 0.01 |
| PEG-2 | 0.39 ± 0.11 | 0.03 ± 0.02 | 0.09 ± 0.05 | 0.01 ± 0.00 |
| PEG-4 | 0.39 ± 0.08 | 0.02 ± 0.00 | 0.06 ± 0.03 | 0.01 ± 0.01 |
| PEG-6 | 0.30 ± 0.16 | 0.03 ± 0.01 | 0.10 ± 0.06 | 0.02 ± 0.01 |
| PEG-8 | 0.30 ± 0.07 | 0.06 ± 0.04 | 0.08 ± 0.03 | 0.02 ± 0.01 |
| Gly-3 | 0.28 ± 0.05 | 0.05 ± 0.01 | 0.07 ± 0.01 | 0.01 ± 0.00 |
| Gly-5 | 0.11 ± 0.01 | 0.02 ± 0.00 | 0.03 ± 0.01 | 0.01 ± 0.01 |
| Ala-3 | 0.28 ± 0.02 | 0.04 ± 0.02 | 0.07 ± 0.02 | 0.01 ± 0.01 |
| Ala-5 | 0.41 ± 0.05 | 0.04 ± 0.02 | 0.07 ± 0.01 | 0.01 ± 0.01 |
| Phe-3 | 0.31 ± 0.03 | 0.12 ± 0.04 | 0.38 ± 0.07 | 0.24 ± 0.05 |
| Phe-5 | 0.32 ± 0.02 | 0.18 ± 0.03 | 0.80 ± 0.06 | 0.89 ± 0.15 |
| Tyr-3 | 0.31 ± 0.03 | 0.04 ± 0.01 | 0.38 ± 0.18 | 0.07 ± 0.07 |
| Tyr-5 | 0.43 ± 0.01 | 0.12 ± 0.02 | 0.27 ± 0.15 | 0.14 ± 0.10 |

* Specific cell uptake was determined by blockage using 500 μM 2-PMPA. Values are expressed as % of applied radioactivity (AR) bound to 10⁵ cells.

### REFERENCES

1. Schafer M, Bauder-Wust U, Leotta K, et al. A dimerized urea-based inhibitor of the prostate-specific membrane antigen for 68Ga-PET imaging of prostate cancer. EJNMMI Res. 2012; 2:23.
2. Eder M, Schafer M, Bauder-Wust U, et al. (68) Ga-Complex lipophilicity and the targeting property of a urea-based PSMA inhibitor for PET imaging. Bioconjug Chem. 2012; 23:688-697.
3. Benesova M, Bauder-Wust U, Schafer M, et al. Linker Modification Strategies To Control the Prostate-Specific Membrane Antigen (PSMA)-Targeting and Pharmacokinetic Properties of DOTA-Conjugated PSMA Inhibitors. J Med Chem. 2016; 59:1761-1775.

1-17. (canceled)

18. A PSMA binding ligand or a pharmaceutically acceptable salt or solvate thereof comprising a PSMA binding motif Q and a chelator residue A, linked via at least one linker L^{AQ} comprising at least one neutral amino acid X₁,
wherein X₁ is selected from the group consisting of Gly, Ala, βAla, Phe, amino hexanoic acid (—NH—(CH₂)₅—C(═O)—) and amino acids comprising at least one —(CH₂—CH₂—O)— group between the N-terminus and the C-terminus, more preferably wherein X₁ is selected from the group consisting of Gly, Ala, βAla, Phe and amino acid residues having the structure —NH—CH₂—CH₂—O—(CH₂—CH₂—O)ₓ₁—(CH₂)_{y1}—C(═O)— with x1 being an integer in the range of from 0 to 15, and y1 being 1 or 2.

19. The PSMA binding ligand of claim 18 or a pharmaceutically acceptable salt or solvate thereof, wherein the PSMA binding ligand has the structure (I):

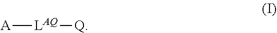

20. The PSMA binding ligand of claim 18 or a pharmaceutically acceptable salt or solvate thereof, wherein the PSMA binding motif Q has the structure: 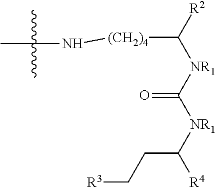
wherein:
R¹ is H or —CH₃, preferably H, and
R², R³ and R⁴ are independently of each other, selected from the group consisting of —CO₂H, —SO₂H, —SO₃H, —OSO₃H, —PO₂H, —PO₃H and —OPO₃H₂.

21. The PSMA binding ligand of claim 18 or a pharmaceutically acceptable salt or solvate thereof,
wherein A is a chelator residue derived from a chelator selected from the group consisting of 1,4,7,10-tetraazacyclododecane-N,N′,N″,N′″-tetraacetic acid (=DOTA), N,N″-bis [2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7,10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl (2-carboxyethyl)phosphinic acid]-4,7-bis [methyl (2-hydroxymethyl)phosphinic acid] (NOPO), 3,6,9,15-tetraazabicyclo [9.3.1]pentadeca-1 (15), 11,13-triene-3,6,9-triacetic acid (=PCTA), N′-{5-[Acetyl(hydroxy)amino]pentyl}-N-[5-({4-[(5-arninopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA).

22. The PSMA binding ligand of claim 18 or a pharmaceutically acceptable salt or solvate thereof, wherein A is a chelator residue having a structure selected from the group consisting of: 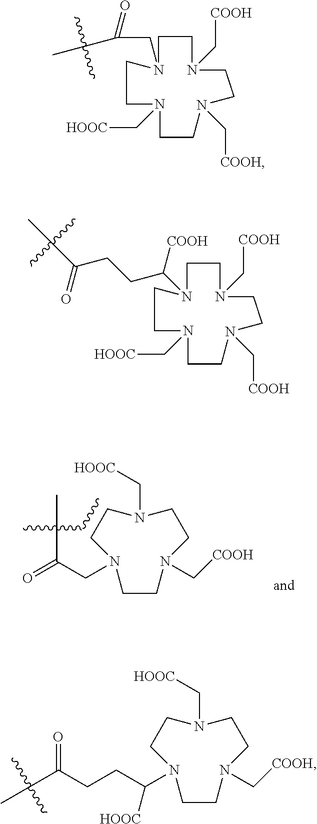
preferably wherein A is a chelator residue having the structure

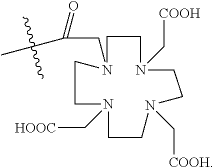 [SMILES: *C(=O)CN1CCN(CC(=O)O)CCN(CC(=O)O)CCN(CC(=O)O)CC1]

23. The PSMA binding ligand of claim 18 or a pharmaceutically acceptable salt or solvate thereof, wherein the linker L^{AQ} comprises at least one amino acid building block AS^{a}, wherein AS^{a} has the structure: 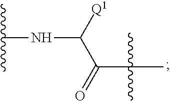
and
wherein Q¹ is selected from the group consisting of alkylaryl, arylalkyl, aryl, alkylheteroaryl, heteroarylalkyl and heteroaryl.

24. The PSMA binding ligand of claim 18 or a pharmaceutically acceptable salt or solvate thereof, wherein the linker L^{AQ} comprises at least one amino acid building block AS^{b}, wherein AS^{b} has the structure (b); 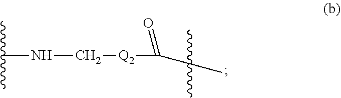
and
wherein Q² is selected from the group consisting of aryl, alkylaryl, arylalkyl, cycloalkyl, heterocycloalkyl, heteroaryl, heteroarylalkyl and alkylheteroaryl, preferably wherein Q² is 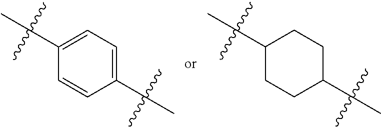 [SMILES: *c1ccc(*)cc1 ; *C1CCC(*)CC1]
more preferably

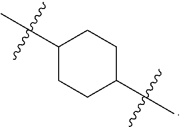 [SMILES: *C1CCC(*)CC1]

25. The PSMA binding ligand of claim 18 or a pharmaceutically acceptable salt or solvate thereof, wherein the PSMA binding ligand has the structure (Ia): 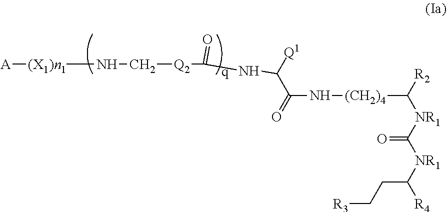
wherein:
R¹ is H or —CH₃, preferably H,
R², R³ and R⁴ are independently of each other, selected from the group consisting of —CO₂H, —SO₂H, —SO₃H, —OSO₃H, —PO₂H, —PO₃H and —OPO₃H₂,
Q¹ is selected from the group consisting of alkylaryl, arylalkyl, aryl, alkylheteroaryl, heteroarylalkyl and heteroaryl,
Q² is selected from the group consisting of aryl, alkylaryl, arylalkyl, cycloalkyl, heterocycloalkyl, heteroaryl, heteroarylalkyl and alkylheteroaryl,
q is an integer of from 0-3, and
n1 is an integer in the range of from 1 to 25.

26. The PSMA binding ligand of claim 18, wherein the linker L^{AQ} comprises the building block (X₁)ₙ₁, and wherein:
—X₁ is selected from the group consisting of Gly, Ala, βAla and Phe and n1 is in the range of from 2 to 20, preferably 3 to 15, more preferably, 3, 5, 10 or 15 or
X₁ has the structure —NH—CH₂—CH₂—O—(CH₂—CH₂—O)ₓ₁—(CH₂)_{y1}—C(═O)— and with integer x1 being in the range of from 0 to 15, and y1 being 1 or 2 and with n1 being 1, or
X₁ has the structure —NH—(CH₂)₅—C(═O)— and n1 is in the range of from 1 to 20, preferably 1 to 4, or
X₁ is Tyr and n1 is in the range of from 2 to 20, preferably 3 to 15, more preferably, 3, 5, 10 or 15.

27. A complex comprising:
(a) a radionuclide, and
(b) the PSMA binding ligand of claim 18 or a pharmaceutically acceptable salt or solvate thereof.

28. The complex of claim 27, wherein the radionuclide is selected from the group consisting of ⁸⁹Zr, ⁴⁴Sc, ¹¹¹In, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe, and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb), more preferably selected from the group consisting ⁹⁰Y, ⁶⁸Ga, ¹⁷⁷Lu, ²²⁵Ac, and ²¹³Bi, more preferably, the radionuclide is ¹⁷⁷Lu or ²²⁵Ac.

29. A pharmaceutical composition comprising the PSMA binding ligand of claim 18.

30. A method for treating and/or preventing a PSMA expressing cancer and/or metastases thereof, comprising administering to a subject in need the complex of claim 27.

31. A method for treating and/or preventing PSMA expressing cancer and/or metastases thereof, in particular prostate cancer and/or metastases thereof, comprising administering to a subject in need the PSMA binding ligand or a pharmaceutically acceptable salt or solvate thereof of claim 18.

32. A method for diagnosing cancer and/or metastases thereof, preferably PSMA-expressing cancer and/or metastases thereof, in particular prostate cancer and/or metastases thereof, comprising using a diagnostic method comprising the complex of claim 27.

33. A method for diagnosing cancer and/or metastases thereof, preferably PSMA-expressing cancer and/or metastases thereof, in particular prostate cancer and/or metastases thereof, comprising using a diagnostic method comprising the PSMA binding ligand or a pharmaceutically acceptable salt or solvate thereof of claim 18.

34. The method of claim 32, wherein the radionuclide is a β-emitter, more preferably ¹⁷⁷Lu and wherein preferably the activity dosage of the complex is at least 100 kBq/kg body weight, more preferably at least 500 kBq/kg body weight, most preferably at least 1 MBq/kg body weight.

35. The method of claim 30, wherein the radionuclide is an α-emitter, more preferably is ²²⁵Ac and wherein preferably the activity dosage of the complex is preferably at least 75 kBq/kg body, more preferably at least 100 kBq/kg body weight.
